(19) United States
(12) Patent Application Publication
(10) Pub. No.: US 2014/0066398 A1
(43) Pub. Date: Mar. 6, 2014

(54) HCV POLYMERASE INHIBITORS

(71) Applicant: Medivir, AB, Huddinge (SE)

(72) Inventors: Anders ENEROTH, Huddinge (SE); Bjorn Klasson, Huddinge (SE); Magnus Nilsson, Huddinge (SE); Pedro Pinho, Huddinge (SE); Bertil Samuelsson, Huddinge (SE); Christian Sund, Huddinge (SE)

(73) Assignee: Medivir, AB, Huddinge (SE)

(21) Appl. No.: 14/051,298

(22) Filed: Oct. 10, 2013

Related U.S. Application Data
(63) Continuation of application No. 13/693,729, filed on Dec. 4, 2012, now abandoned.

(30) Foreign Application Priority Data
Dec. 5, 2011 (SE) 1151157-3

(51) Int. Cl. C07H 19/10; A61K 45/06; A61K 31/7072

(52) U.S. Cl. CPC C07H 19/10; A61K 31/7072; A61K 45/06
USPC 514/51; 536/26.8

(57) ABSTRACT

The invention provides compounds of the formula:

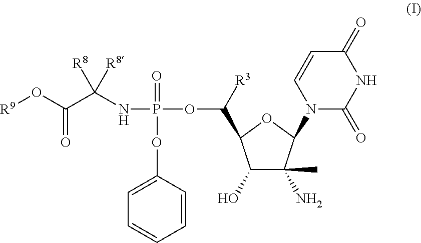

which are of use in the treatment or prophylaxis of hepatitis C virus infection, and related aspects.

### TECHNICAL FIELD

The present invention relates to inhibitors of the polymerase of hepatitis C virus (HCV), prodrugs thereof and their use in the treatment or prophylaxis of HCV infection.

### BACKGROUND OF THE INVENTION

HCV is a single stranded, positive-sense RNA virus belonging to the Flaviviridae family of viruses in the hepacivirus genus. The NS5B region of the RNA polygene encodes an RNA dependent RNA polymerase (RdRp), which is essential to viral replication. Following the initial acute infection, a majority of infected individuals develop chronic hepatitis because HCV replicates preferentially in hepatocytes but is not directly cytopathic. In particular, the lack of a vigorous T-lymphocyte response and the high propensity of the virus to mutate appear to promote a high rate of chronic infection. Chronic hepatitis can progress to liver fibrosis, leading to cirrhosis, end-stage liver disease and HCC (hepatocellular carcinoma), making it the leading cause of liver transplantations.

There are six major HCV genotypes and more than 50 subtypes, which are differently distributed geographically. HCV genotype 1 is the predominant genotype in Europe and in the US. The extensive genetic heterogeneity of HCV has important diagnostic and clinical implications, perhaps explaining difficulties in vaccine development and the lack of response to current therapy.

Transmission of HCV can occur through contact with contaminated blood or blood products, for example following blood transfusion or intravenous drug use. The introduction of diagnostic tests used in blood screening has led to a downward trend in post-transfusion HCV incidence. However, given the slow progression to the end-stage liver disease, the existing infections will continue to present a serious medical and economic burden for decades.

Current HCV therapies are based on (pegylated) interferon-alpha (IFN-α) in combination with ribavirin. This combination therapy yields a sustained virologic response in more than 40% of patients infected by genotype 1 viruses and about 80% of those infected by genotypes 2 and 3. Beside the limited efficacy on HCV genotype 1, this combination therapy has significant side effects and is poorly tolerated in many patients. Major side effects include influenza-like symptoms, hematologic abnormalities and neuropsychiatric symptoms. Hence there is a need for more effective, convenient and better-tolerated treatments.

Experience with HIV drugs, in particular with HIV protease inhibitors, has taught that sub-optimal pharmacokinetics and complex dosing regimes quickly result in inadvertent compliance failures. This in turn means that the 24 hour trough concentration (minimum plasma concentration) for the respective drugs in an HIV regime frequently falls below the IC₉₀ or ED₉₀ threshold for large parts of the day. It is considered that a 24 hour trough level of at least the IC₅₀, and more realistically, the IC₉₀ or ED₉₀, is essential to slow down the development of drug escape mutants. Achieving the necessary pharmacokinetics and drug metabolism to allow such trough levels provides a stringent challenge to drug design.

The NS5B RdRp is absolutely essential for replication of the single-stranded, positive sense HCV RNA genome which makes it an attractive target for the development of antiviral compounds. There are two major classes of NS5B inhibitors: non-nucleoside inhibitors (NNIs) and nucleoside analogues. The NNIs bind to allosteric regions of the protein whereas the nucleoside inhibitors are anabolized to the corresponding nucleotide and act as alternative substrate for the polymerase. The formed nucleotide is then incorporated in the nascent RNA polymer chain and can terminate the growth of the polymer chain. To date, both nucleoside and non-nucleoside inhibitors of NS5B are known.

As stated above, the inhibition mechanism of nucleoside inhibitors involves phosphorylation of the nucleoside to the corresponding triphosphate. The phosphorylation is commonly mediated by host cell kinases and is an absolute requirement for the nucleoside to be active as an alternative substrate for the NS5B polymerase. Typically, the first phosphorylation step, i.e. conversion of the nucleoside to the nucleoside 5′-monophosphate is the rate limiting step. Subsequent conversion of the monophosphate to the di- and tri-phosphate usually proceed facile and are usually not rate limiting. A strategy for increasing nucleoside triphosphate production is to use cell permeable nucleoside prodrugs of the monophosphate, i.e. a nucleoside carrying a masked phosphate moiety, a “prodrug moiety”, which are susceptible to intracellular enzymatic activation leading to a nucleoside monophosphate. The thus formed monophosphate is subsequently converted to the active triphosphate by cellular kinases.

Chemical modifications of an active compound to afford a potential prodrug produces an entirely new molecular entity which can exhibit undesirable physical, chemical and biological properties, thus the identification of optimal prodrugs remains an uncertain and challenging task.

There is a need for HCV inhibitors that may overcome the disadvantages of current HCV therapy such as side effects e.g. toxicity, limited efficacy, the emerging of resistance, and compliance failures, as well as improve the sustained viral response.

The present invention provides new of HCV inhibiting compounds which have useful properties regarding one or more of the following parameters: antiviral efficacy; favourable profile of resistance development; lack of toxicity and genotoxicity; favourable pharmacokinetics and pharmacodynamics; and ease of formulation and administration. The skilled person will appreciate that an HCV inhibiting compound of the present invention need not demonstrate an improvement in every respect over all known compounds but may instead provide a balance of properties which in combination mean that the HCV inhibiting compound is a valuable alternative pharmaceutical agent.

Compounds of the invention may also be attractive due to the fact that they lack activity against other viruses, i.e. are selective, in particular against HIV. HIV infected patients often suffer from co-infections such as HCV. Treatment of such patients with an HCV inhibitor that also inhibits HIV may lead to the emergence of resistant HIV strains.

### DESCRIPTION OF THE INVENTION

The present invention provides compounds represented by formula I:

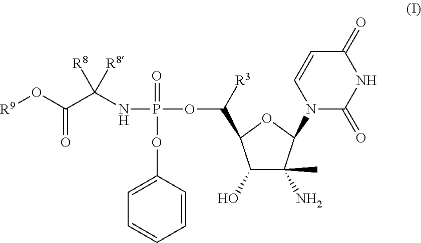

wherein:
R³ is H or CH₃;
R⁸ and R^{8′} are each independently selected from H, C₁-C₆alkyl and benzyl; or R⁸ and R^{8′} together with the carbon atom to which they are attached from a C₃-C₇cycloalkylene group;
R⁹ is C₁-C₁₀alkyl, C₁-C₁₀haloalkyl, C₃-C₇cycloalkyl, benzyl or phenyl, any of which is optionally substituted with 1, 2 or 3 substituents each independently selected from hydroxy,
C₁-C₆alkoxy, amino, mono- and di-C₁-C₆alkylamino;
   or a pharmaceutically acceptable salt and/or solvate thereof.

The compounds of formula I may optionally be provided in the form of a pharmaceutically acceptable salt and/or solvate. In one embodiment the compound of formula I is provided in the form of a pharmaceutically acceptable salt. In a second embodiment the compound of formula I is provided in the form of a pharmaceutically acceptable solvate. In a third embodiment the compound of formula I is provided in its free form.

In certain embodiments R³ is CH₃. R³ is typically H.

Typical configurations for R⁹ include C₁-C₆alkyl. Of particular interest are C₁-C₄alkyl, especially methyl, ethyl, isopropyl, isobutyl and tert-butyl. Further typical configurations for R⁹ include phenyl and benzyl, especially benzyl.

Typically, the moiety —NH—C(R⁸)(R^{8′})—C(═O)— forms an amino acid residue, including natural and non-natural amino acid residues. Typically one of R⁸ and R^{8′} is hydrogen, and the other is hydrogen or C₁-C₆alkyl, such as isopropyl or isobutyl. Of particular interest are amino acid residues wherein R^{8′} is hydrogen, examples are glycine, (Gly) alanine (Ala), valine (Val), isoleucine (Ile) and phenylalanine (Phe) residues, i.e., R^{8′} is H and R⁸ is methyl, isopropyl, isobutyl or benzyl respectively. In compounds wherein R^{8′} is hydrogen and R⁸ is other than hydrogen, the configuration at the asymmetric carbon atom is typically that of an L-amino acid, in particular L-Ala, L-Val, L-Ile, and L-Phe.

In a typical configuration, one of R⁸ and R^{8′} is H and the other is or methyl, or R⁸ and R^{8′} are both methyl.

In a further configuration, R⁸ and R^{8′} together with the carbon atom to which they are attached form C₃-C₇cycloalkyl, for example cyclopropyl or cyclobutyl.

In a typical configuration of the group (ii) R⁷ is phenyl, R⁸ is H, R^{8′} is C₁-C₃alkyl (such as methyl, ethyl or isopropyl), and R⁹ is C₁-C₆alkyl or C₃-C₇cycloalkyl (such as cyclopropyl, cyclobutyl or cyclopentyl).

In one embodiment of the invention, R⁹ is cyclopropyl.

In another embodiment of the invention, R⁹ is cyclobutyl.

In another embodiment of the invention, R⁹ is cyclopentyl.

In another embodiment of the invention, R⁹ is cyclohexyl.

In another embodiment of the invention, R⁹ is cycloheptyl.

In another embodiment of the invention, R⁹ is cyclooctyl.

In another embodiment of the invention, R⁹ is methyl.

In another embodiment of the invention, R⁹ is isopropyl.

In another embodiment of the invention, R⁹ is isobutyl.

In another embodiment of the invention, R⁹ is n-propyl.

In another embodiment of the invention, R⁹ is n-pentyl.

In another embodiment of the invention, R⁹ is n-butyl.

In another embodiment of the invention, R⁹ is 2-ethylbutyl.

In another embodiment of the invention, R⁹ is 2-propylpentyl.

In another embodiment of the invention, R⁹ is sec. butyl

In another embodiment of the invention, R⁹ is 2,2-dimethylpropyl.

In another embodiment of the invention, R⁹ is 3,3-dimethylbutyl.

In another embodiment of the invention, R⁹ is cyclopropylmethyl.

In another embodiment of the invention, R⁹ is (S)-pentan-2-yl

In another embodiment of the invention, R⁹ is (R)-pentan-2-yl.

In another embodiment of the invention, R⁹ is pentan-3-yl.

In another embodiment of the invention, R⁹ is cyclobutylmethyl.

In another embodiment of the invention, R⁹ is cyclopentylmethyl.

When administered in vivo, the compounds of the invention will be metabolised and transformed into the triphosphate i.e. a compound having the structure:

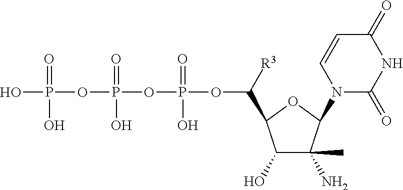

or a pharmaceutically acceptable salt thereof, such as the potassium salt or the sodium salt.

Consequently, there is provided a compound of formula I for use as a medicament, in particular for use in the treatment or prophylaxis of HCV infection, especially the treatment of HCV infection.

Further provided is the use of a compound of formula I in the manufacture of a medicament, in particular a medicament for the treatment or prophylaxis of HCV infection, especially a medicament for the treatment of HCV infection.

Additionally, there is provided a method for the treatment or prophylaxis of HCV infection comprising the administration of a compound of formula I, in particular a method for the treatment of HCV infection comprising the administration of a compound of formula I.

In a further aspect, the invention concerns the use of the compounds of the invention for inhibiting HCV.

Additionally, there is provided the use of the compounds of formula I for the treatment or prophylaxis of HCV infection, such as the treatment or prophylaxis of HCV infection in humans. In a preferred aspect, the invention provides the use of compounds of formula I for the treatment of HCV infection, such as the treatment of HCV infection in humans.

Furthermore, the invention relates to a method for manufacturing compounds of formula I, to novel intermediates of use in the manufacture of compounds of formula I and to the manufacture of such intermediates.

In a further aspect, the invention provides pharmaceutical compositions comprising a compound of formula I in association with a pharmaceutically acceptable adjuvant, diluent, excipient or carrier. The pharmaceutical composition will typically contain an antivirally effective amount (e.g. for humans) of the compound of formula I, although sub-therapeutic amounts of the compound of formula I may nevertheless be of value when intended for use in combination with other agents or in multiple doses.

The skilled person will recognise that references to compounds of formula I will include any subgroup of the compounds of formula I described herein.

Representative HCV genotypes in the context of treatment or prophylaxis in accordance with the invention include genotype 1b (prevalent in Europe) and 1a (prevalent in North America). The invention also provides a method for the treatment or prophylaxis of HCV infection, in particular of the genotype 1a or 1b. Typically, the invention provides a method for the treatment of HCV infection, in particular of the genotype 1a or 1b.

The compounds of formula I are represented as a defined stereoisomer. The absolute configuration of such compounds can be determined using art-known methods such as, for example, X-ray diffraction or NMR and/or implication from start materials of known stereochemistry. Pharmaceutical compositions in accordance with the invention will preferably comprise substantially stereoisomerically pure preparations of the indicated stereoisomer.

Pure stereoisomeric forms of the compounds and intermediates as mentioned herein are defined as isomers substantially free of other enantiomeric or diastereomeric forms of the same basic molecular structure of said compounds or intermediates. In particular, the term “stereoisomerically pure” concerns compounds or intermediates having a stereoisomeric excess of at least 80% (i.e. minimum 90% of one isomer and maximum 10% of the other possible isomers) up to a stereoisomeric excess of 100% (i.e. 100% of one isomer and none of the other), more in particular, compounds or intermediates having a stereoisomeric excess of 90% up to 100%, even more in particular having a stereoisomeric excess of 94% up to 100% and most in particular having a stereoisomeric excess of 97% up to 100%. The terms “enantiomerically pure” and “diastereomerically pure” should be understood in a similar way, but then having regard to the enantiomeric excess, and the diastereomeric excess, respectively, of the mixture in question.

Pure stereoisomeric forms of the compounds and intermediates of this invention may be obtained by the application of art-known procedures. For instance, enantiomers may be separated from each other by the selective crystallization of their diastereomeric salts with optically active acids or bases. Examples thereof are tartaric acid, dibenzoyltartaric acid, ditoluoyltartaric acid and camphorsulfonic acid. Alternatively, enantiomers may be separated by chromatographic techniques using chiral stationary phases. Said pure stereochemically isomeric forms may also be derived from the corresponding pure stereochemically isomeric forms of the appropriate starting materials, provided that the reaction occurs stereospecifically. Preferably, if a specific stereoisomer is desired, said compound is synthesized by stereospecific methods of preparation. These methods will advantageously employ enantiomerically pure starting materials.

The diastereomeric racemates of the compounds of formula I can be obtained separately by conventional methods. Appropriate physical separation methods that may advantageously be employed are, for example, selective crystallization and chromatography, e.g. column chromatography.

The present invention also includes isotope-labelled compounds of formula I or any subgroup of formula I, wherein one or more of the atoms is replaced by an isotope of that atom, i.e. an atom having the same atomic number as, but an atomic mass different from, the one(s) typically found in nature. Examples of isotopes that may be incorporated into the compounds of formula I or any subgroup of formula I, include but are not limited to isotopes of hydrogen, such as ²H and ³H (also denoted D for deuterium and T for tritium, respectively), carbon, such as ¹¹C, ¹³C and ¹⁴C, nitrogen, such as ¹³N and ¹⁵N, oxygen, such as ¹⁵O, ¹⁷O and ¹⁸O, phosphorus, such as ³¹P and ³²P, sulphur, such as ³⁵S, fluorine, such as ¹⁸F, chlorine, such as ³⁶Cl, bromine such as ⁷⁵Br, ⁷⁶Br, ⁷⁷Br and ⁸²Br, and iodine, such as ¹²³I, ¹²⁴I, ¹²⁵I and ¹³¹I. The choice of isotope included in an isotope-labelled compound will depend on the specific application of that compound. For example, for drug or substrate tissue distribution assays, compounds wherein a radioactive isotope such as ³H or ¹⁴C is incorporated will generally be most useful. For radio-imaging applications, for example positron emission tomography (PET) a positron emitting isotope such as ¹¹C, ¹⁸F, ¹³N or ¹⁵O will be useful. The incorporation of a heavier isotope, such as deuterium, i.e. ²H, may provide greater metabolic stability to a compound of formula I or any subgroup of formula I, which may result in, for example, an increased in vivo half life of the compound or reduced dosage requirements.

Isotope-labelled compounds of formula I or any subgroup of formula I can be prepared by processes analogous to those described in the Schemes and/or Examples herein below by using the appropriate isotope-labelled reagent or starting material instead of the corresponding non-isotope-labelled reagent or starting material, or by conventional techniques known to those skilled in the art.

The pharmaceutically acceptable addition salts comprise the therapeutically active non-toxic acid and base addition salt forms of the compounds of formula I. Of interest are the free, i.e. non-salt forms of the compounds of formula I.

The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propionic, hydroxyacetic, lactic, pyruvic, oxalic (i.e. ethanedioic), malonic, succinic (i.e. butanedioic acid), maleic, fumaric, malic (i.e. hydroxylbutanedioic acid), tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds of formula I containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The term “solvates” covers any pharmaceutically acceptable solvates that the compounds of formula I as well as the salts thereof, are able to form. Such solvates are for example hydrates, alcoholates, e.g. ethanolates, propanolates, and the like, especially hydrates.

Some of the compounds of formula I may also exist in their tautomeric form. For example, tautomeric forms of amide groups (—C(═O)—NH—) are iminoalcohols (—C(OH)═N—), which can become stabilized in rings with aromatic character. Such forms, although not explicitly indicated in the structural formulae represented herein, are intended to be included within the scope of the present invention.

As used herein, the following terms have the meanings as defined below, unless otherwise noted:

“Cₘ-Cₙalkyl” on its own or in composite expressions such as Cₘ-Cₙhaloalkyl, Cₘ-Cₙalkylcarbonyl, Cₘ-Cₙalkylamine, etc. represents a straight or branched alkyl radical having the number of carbon atoms designated, e.g. C₁-C₄alkyl means an alkyl radical having from 1 to 4 carbon atoms. C₁-C₆alkyl has a corresponding meaning, including also all straight and branched chain isomers of pentyl and hexyl. Preferred alkyl radicals for use in the present invention are C₁-C₆alkyl, including methyl, ethyl, n-propyl, isopropyl, n-butyl, isobutyl, sec-buty, tert-butyl, n-pentyl and n-hexyl, especially C₁-C₄alkyl such as methyl, ethyl, n-propyl, isopropyl, t-butyl, n-butyl and isobutyl. Methyl and isopropyl are typically preferred.

The term “Cₘ-Cₙhaloalkyl” as used herein represents Cₘ-Cₙalkyl wherein at least one C atom is substituted with a halogen (e.g. the Cₘ-Cₙhaloalkyl group may contain one to three halogen atoms), preferably chloro or fluoro. Typical haloalkyl groups are C₁-C₂haloalkyl, in which halo suitably represents fluoro. Exemplary haloalkyl groups include fluoromethyl, difluoromethyl and trifluoromethyl.

The term “Cₘ-Cₙhydroxyalkyl” as used herein represents Cₘ-Cₙalkyl wherein at least one C atom is substituted with one hydroxy group. Typical Cₘ-Cₙhydroxyalkyl groups are Cₘ-Cₙalkyl wherein one C atom is substituted with one hydroxy group. Exemplary hydroxyalkyl groups include hydroxymethyl and hydroxyethyl.

The term “Cₘ-Cₙaminoalkyl” as used herein represents Cₘ-Cₙalkyl wherein at least one C atom is substituted with one amino group. Typical Cₘ-Cₙaminoalkyl groups are Cₘ-Cₙalkyl wherein one C atom is substituted with one amino group. Exemplary aminoalkyl groups include aminomethyl and aminoethyl.

The term “Cₘ-Cₙalkylene” as used herein represents a straight or branched divalent alkyl radical having the number of carbon atoms indicated. Preferred Cₘ-Cₙalkylene radicals for use in the present invention are C₁-C₃alkylene i.e. methylene, ethylene and propylene.

The term “Me” means methyl, and “MeO” means methoxy.

The term “Cₘ-Cₙalkylcarbonyl” represents a radical of the formula Cₘ-Cₙalkyl-C(═O) wherein the Cₘ-Cₙalkyl moiety is as defined above. Typically, “Cₘ-Cₙalkylcarbonyl” is C₁-C₆alkyl-C(═O).

“Cₘ-Cₙalkoxy” represents a radical Cₘ-Cₙalkyl-O— wherein Cₘ-Cₙalkyl is as defined above. Of particular interest is C₁-C₄alkoxy which includes methoxy, ethoxy, n-propoxy, isopropoxy, t-butoxy, n-butoxy and isobutoxy. Methoxy and isopropoxy are typically preferred. C₁-C₆alkoxy has a corresponding meaning, expanded to include all straight and branched chain isomers of pentoxy and hexoxy.

The term “Cₘ-Cₙalkoxycarbonyl” represents a radical of the formula Cₘ-Cₙalkoxy-C(═O)— wherein the Cₘ-Cₙalkoxy moiety is as defined above. Typically, “Cₘ-Cₙalkoxycarbonyl” is C₁-C₆alkoxy-C(═O).

The term “amino” represents the radical —NH₂.

The term “halo” represents a halogen radical such as fluoro, chloro, bromo or iodo. Typically, halo groups are fluoro or chloro.

The term “aryl” means a phenyl, biphenyl or naphthyl group.

The term “heterocycloalkyl” represents a stable saturated monocyclic 3-7 membered ring containing 1-3 heteroatoms independently selected from O, S and N. In one embodiment of the invention the stable saturated monocyclic 3-7 membered ring contains 1 heteroatom selected from O, S and N. In a second embodiment of the invention the stable saturated monocyclic 3-7 membered ring contains 2 heteroatoms independently selected from O, S and N. In a third embodiment the stable saturated monocyclic 3-7 membered ring contains 3 heteroatoms independently selected from O, S and N. The stable saturated monocyclic 3-7 membered ring containing 1-3 heteroatoms independently selected from O, S and N may typically be a 5-7 membered ring, such as a 5 or 6 membered ring.

The term “heteroaryl” represents a stable mono or bicyclic aromatic ring system containing 1-4 heteroatoms independently selected from O, S and N, each ring having 5 or 6 ring atoms. In one embodiment of the invention the stable mono or bicyclic aromatic ring system contains one heteroatom selected from O, S and N, each ring having 5 or 6 ring atoms. In a second embodiment of the invention the stable mono or bicyclic aromatic ring system contains two heteroatoms independently selected from O, S and N, each ring having 5 or 6 ring atoms. In a third embodiment the stable mono or bicyclic aromatic ring system contains three heteroatoms independently selected from O, S and N, each ring having 5 or 6 ring atoms. In a fourth embodiment the stable mono or bicyclic aromatic ring system contains four heteroatoms independently selected from O, S and N, each ring having 5 or 6 ring atoms.

The term “C₃-Cₙcycloalkyl” represents a cyclic monovalent alkyl radical having the number of carbon atoms indicated, e.g. C₃-C₇cycloalkyl means a cyclic monovalent alkyl radical having from 3 to 7 carbon atoms. Preferred cycloalkyl radicals for use in the present invention are C₃-C₄alkyl i.e. cyclopropyl and cyclobutyl.

The term “aminoCₘ-Cₙalkyl” represents a Cₘ-Cₙalkyl radical as defined above which is substituted with an amino group, i.e. one hydrogen atom of the alkyl moiety is replaced by an NH₂-group. Typically, “aminoCₘ-Cₙalkyl” is aminoC₁-C₆alkyl.

The term “aminoCₘ-Cₙalkylcarbonyl” represents a Cₘ-Cₙalkylcarbonyl radical as defined above, wherein one hydrogen atom of the alkyl moiety is replaced by an NH₂-group. Typically, “aminoCₘ-Cₙalkylcarbonyl” is aminoC₁-H₆alkylcarbonyl. Examples of aminoCₘ-Cₙalkylcarbonyl include but are not limited to glycyl: C(═O)CH₂NH₂, alanyl: C(═O)CH(NH₂)CH₃, valinyl: C═OCH(NH₂)CH(CH₃)₂, leucinyl: C(═O)CH(NH₂)(CH₂)₃CH₃, isoleucinyl: C(═O)CH(NH₂)CH(CH₃)(CH₂CH₃) and norleucinyl: C(═O)CH(NH₂)(CH₂)₃CH₃ and the like. This definition is not limited to naturally occurring amino acids.

Related terms, are to be interpreted accordingly in line with the definitions provided above and the common usage in the technical field.

As used herein, the term “(═O)” forms a carbonyl moiety when attached to a carbon atom. It should be noted that an atom can only carry an oxo group when the valency of that atom so permits.

The term “monophosphate, diphosphate and triphosphate ester” refers to groups:

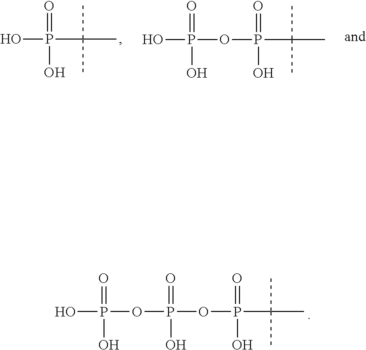

As used herein, the radical positions on any molecular moiety used in the definitions may be anywhere on such a moiety as long as it is chemically stable. When any variable is present occurs more than once in any moiety, each definition is independent.

Whenever used herein, the term “compounds of formula I”, or “the present compounds” or similar terms, it is meant to include the compounds of formula I and subgroups of compounds of formula I, including the possible stereochemically isomeric forms, and their pharmaceutically acceptable salts and solvates.

In general, the names of compounds used in this application are generated using ChemDraw Ultra 12.0. In addition, if the stereochemistry of a structure or a portion of a structure is not indicated with for example bold or dashed lines, the structure or portion of that structure is to be interpreted as encompassing all stereoisomers of it.

### General Synthetic Methods

Compounds of the present invention may be prepared by a variety of methods e.g. as depicted in the illustrative synthetic schemes shown and described below. The starting materials and reagents used are available from commercial suppliers or can be prepared according to literature procedures set forth in references using methods well known to those skilled in the art.

Scheme 1 illustrates a route to the parent nucleoside 1 h, i.e. the compound having a hydroxy group in the 5′-position.

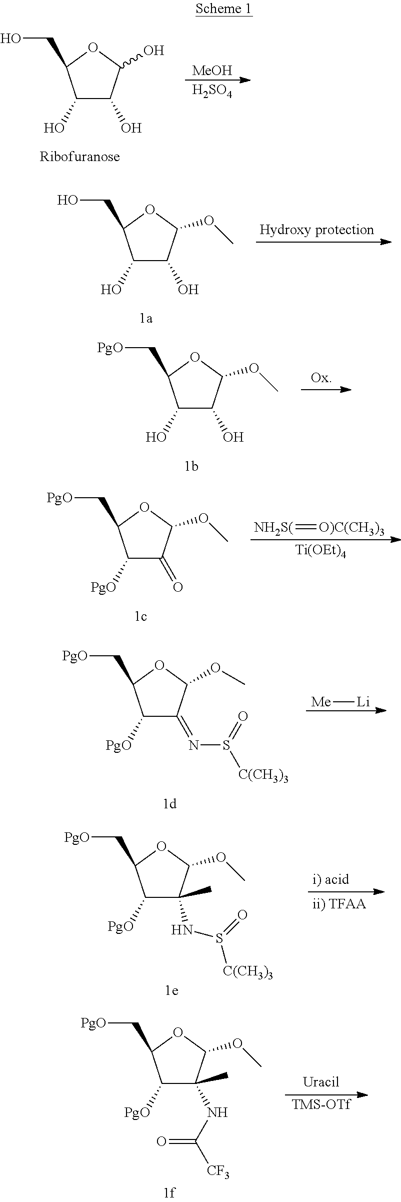

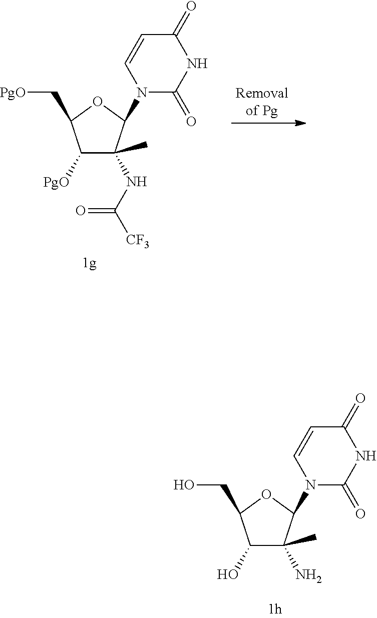

The 2′-hydroxy compound (1b) can be obtained from ribofuranoside by first preparing the methyl glycoside using standard conditions such as treatment with methanol under acidic conditions, followed by an appropriate protecting group strategy. For example, a cyclic protecting group can be used to protect the 3′- and 6′-hydroxy groups and leave the 2′-hydroxy group unprotected. A suitable protecting group for this purpose is for instance a cyclic disiloxane like 1,1,3,3-tetraisopropyl-1,3-disiloxane or a cyclic acetal like 2,2-dimethyl-1,3-dioxolane. Alternatively, the 2′-, 3′- and 5′-hydroxy groups may be all protected at the same time using for instance a benzyl protecting group or the like whereafter the 2′-group is selectively removed, effected by treatment with tin tetrachloride. The 2′-hydroxy compound is then oxidized using any convenient oxidation method, such as oxidation with Dess Martin periodinane or pyridinium dichromate (PDC) or TEMPO optionally in the presence of the co-oxidant [(diacethoxy)iodo]benzene (BAIB), and the thus afforded 2′-oxo derivative (1c) is reacted with 2-methyl-2-propane sulphonamide in the presence of titanium tetraethoxide to provide the sulphinylamide derivative (1d). Introduction of the desired 2-Me group may now be performed using any suitable alkylation method. An organometallic reagent such as a Grignard reagent or an organolithium, organocuprate, organozinc reagent or the like may be used. Suitable conditions are for instance using MeMgBr in an ethereal solvent such as THF, or methyllithium in THF or the like. Prior to introduction of uracil, the sulphinyl group is preferably replaced with a more acid stable N-protecting group for instance a trifluoroacetyl group, accordingly, the sulphinyl derivative (1e) is treated with acid, e.g. HCl in dioxane or the like, followed by acylation of the liberated amine effected by treatment with an acylating agent like trifluoroacetic acid anhydride (TFAA). The suitably protected methyl glycoside (1f) is then condensed with uracil using standard methods for nucleoside formation, such as reaction with silylated uracil in the presence of a Lewis acid such as SnCl₄ or trimethylsilyl trifluoromethanesulphonate (TMS-OTf) in an inert solvent like acetonitrile, to provide the nucleoside (1g). Removal finally of the protecting groups using the suitable conditions according to the protecting group used, provides the unprotected nucleoside (1h). Typically, the trifluoroacetyl group is removed under basic conditions, such as treatment with sodium hydroxide and sodium carbonate in methanol or equivalent.

As will be obvious for a person skilled in the art, the choice of hydroxy protecting groups should be done in relation to the subsequent reactions steps to be performed.

The afforded nucleoside (1h) can then be transformed into a 5′-mono, di- or tri-phosphate or to a prodrug using any of the methods described herein below, or it may be further transformed to provide additional compounds of the invention.

Compounds of the invention wherein R³ is methyl can be prepared by protecting the 2′-amino function of compound 1h and subsequently proceed as illustrated in Scheme 2.

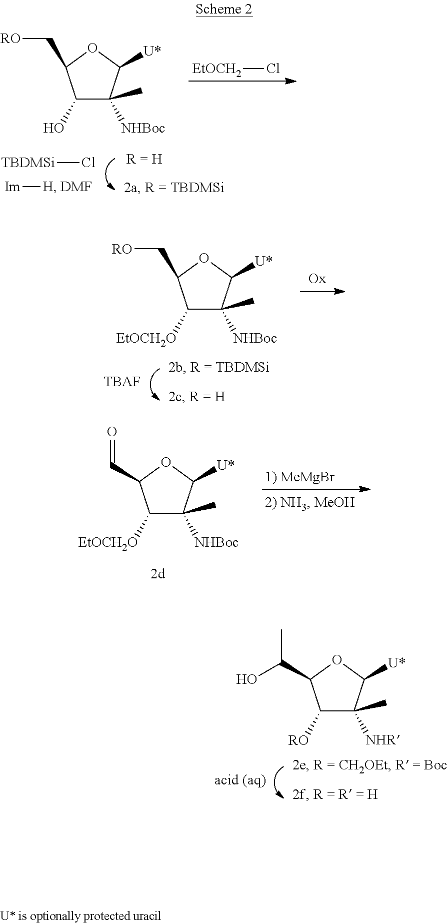

In order to introduce a methyl group in the 5′-position, a protecting group strategy leading to a 5′-unprotected-3′-protected compound is required. The primary 5′-hydroxy group can be selectively protected with for instance a silyl group such as a tert.butyldimethylsilyl group by treatment with the appropriate silylating agent such as the silyl chloride in the presence of imidazole or equivalent. Subsequent protection of the 3′-hydroxy group with for example an alkoxy alkyl ether such as ethoxy methyl ether or the like introduced by reaction with the corresponding chloroalkyl alkyl ether in the presence of a base such as a trialkylamine like DIPEA or similar, or with an acetal protecting group such as tetrahydropyranyl or 2-methoxyisopropyl or the like introduced by reaction with 3,4-dihydro-2H-pyran or 2-methoxyproen respectively in the presence of an acid such as pyridinium p-toluenesulphonate or equivalent, and removal finally of the 5′-O-protecting group effected by treatment with TBAF in case of a TBDMSi group, provides the 5′-hydroxy compound (2c). Oxidation of the primary alcohol using conditions like sodium periodinate or Dess Martin periodinane or any other suitable oxidation method, to the intermediate 5′-aldehyde (2d) followed by introduction of the methyl group effected for instance by a Grignard reaction using Me-Mg—Br, or reaction with methyllithium, provides the 5′-methyl derivative. As the skilled person will realize, alternative organometallic reagents for the introduction of the 5′-methyl group may be used, such as an organocuprate or organozinc reagent. Removal of the N-protecting group on the base using the appropriate conditions according to the protecting groups used, for instance, in the case of a benzoate, treatment with base such as ammonia in methanol or the like provides the 5′-hydroxy compound (2e). The afforded compound is then suitable for introduction of a mono-, di- or triphosphate or a prodrug moiety at the 5′-position to yield a nucleotide or a 5′-nucleoside prodrug respectively, or alternatively, the 3′- and amino-protecting groups can be removed by treatment with acid such as with HCl in THF or methanol, or with TFA in CH₂Cl₂ or the like, to yield the 3′,5′-dihydroxy derivative (2f).

For the preparation of compounds of the invention wherein R³ is H and R⁴ is a phosphoramidate, i.e. a prodrug moiety of formula (ii), advantage can be taken of the higher reactivity of the primary 5′-hydroxy group compared to the secondary 3-hydroxy group, and the phosphoramidate can be introduced directly on the 3′,5-diol without need of any special protecting group strategy. This method is illustrated in Scheme 3.

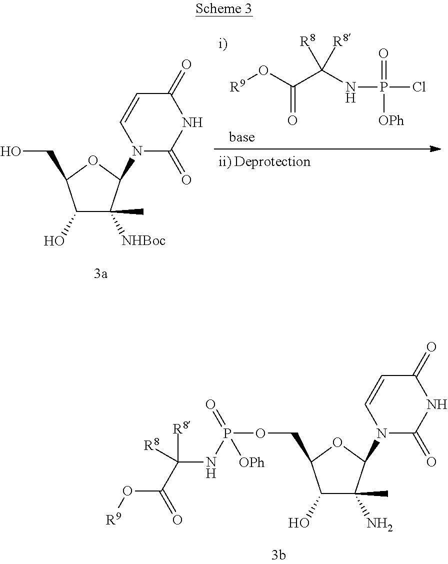

Condensation of nucleoside derivative (3a), prepared as described above, with a desired chlorophosphoramidate in an inert solvent such as an ether, e.g. diethyl ether or THF, or a halogenated hydrocarbon, e.g. dichloromethane, in the presence of a base such as a N-methylimidazole (NMI) or the like, followed by removal of Boc group and the 3′-hydroxy protecting group using standard conditions, provides the phosphoramidate derivative (3b). Compounds of formula I wherein R³ is CH₃ will be achieved using the same strategy but starting from the 3′-protected derivative (2e).

The chlorophosphoramidate used in the above scheme can be prepared in a two-step reaction starting from phosphorus oxychloride (POCl₃), the thus formed phosphorus ester is then further reacted with desired amine. Scheme 4 illustrates the preparation of chlorophosphoramidates.

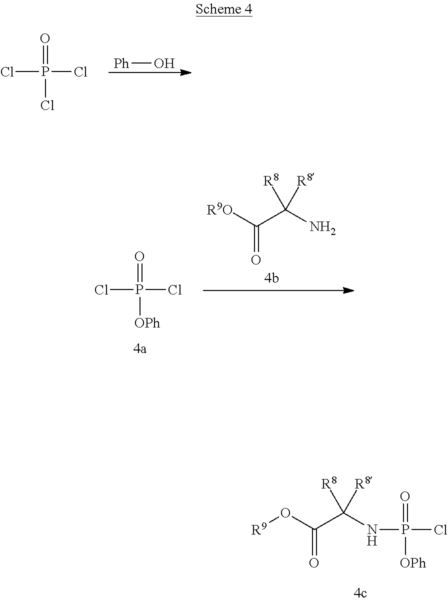

Condensation of POCl₃ with a desired alcohol R⁷OH in an inert solvent like Et₂O provides phenoxy phosphorodichloridate (4a). Subsequent reaction with an amino acid derivative (4b) then provides the chlorophosphoramidate (4c).

The use of various protecting groups (PG) used in schemes above are known to the skilled person, and their utility and further alternatives are extensively described in the literature, see for instance Greene T. W., Wuts P. G. M.: Protective groups in organic synthesis, 2nd ed. New York: Wiley; 1995.

The term “N-protecting group” or “N-protected” as used herein refers to those groups intended to protect the N-terminus of an amino acid or peptide or to protect an amino group against undesirable reactions during synthetic procedures. Commonly used N-protecting groups are disclosed in Greene. N-protecting groups include acyl groups such as formyl, acetyl, propionyl, pivaloyl, t-butylacetyl, 2-chloroacetyl, 2-bromoacetyl, trifluoracetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl, and the like; carbamate forming groups such as benzyloxycarbonyl, p-chlorobenzyloxy-carbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenyl)-1-methylethoxycarbonyl, α,α-dimethyl-3,5-dimethoxybenzyloxycarbonyl, benzhydryloxycarbonyl, t-butoxycarbonyl, diisopropylmethoxycarbonyl, isopropyloxycarbonyl, ethoxycarbonyl, methoxycarbonyl, allyloxycarbonyl, 2,2,2-trichloroethoxycarbonyl, phenoxycarbonyl, 4-nitrophenoxycarbonyl, fluorenyl-9-methoxycarbonyl, cyclopentyloxycarbonyl, adamantyloxycarbonyl, cyclohexyloxycarbonyl, phenylthiocarbonyl, and the like; alkyl groups such as benzyl, triphenylmethyl, benzyloxymethyl and the like; and silyl groups such as trimethylsilyl and the like. Favoured N-protecting groups include formyl, acetyl, benzoyl, pivaloyl, t-butylacetyl, phenylsulfonyl, benzyl (Bz), t-butoxycarbonyl (BOC) and benzyloxycarbonyl (Cbz).

Hydroxy and/or carboxy protecting groups are also extensively reviewed in Greene ibid and include ethers such as methyl, substituted methyl ethers such as methoxymethyl, methylthiomethyl, benzyloxymethyl, t-butoxymethyl, 2-methoxyethoxymethyl and the like, silyl ethers such as trimethylsilyl (TMS), t-butyldimethylsilyl (TBDMS) tribenzylsilyl, triphenylsilyl, t-butyldiphenylsilyl, triisopropyl silyl and the like, substituted ethyl ethers such as 1-ethoxymethyl, 1-methyl-1-methoxyethyl, t-butyl, allyl, benzyl, p-methoxybenzyl, diphenylmethyl, triphenylmethyl and the like, aralkyl groups such as trityl, and pixyl (9-hydroxy-9-phenylxanthene derivatives, especially the chloride). Ester hydroxy protecting groups include esters such as formate, benzylformate, chloroacetate, methoxyacetate, phenoxyacetate, pivaloate, adamantoate, mesitoate, benzoate and the like. Carbonate hydroxy protecting groups include methyl vinyl, allyl, cinnamyl, benzyl and the like.

In one aspect, the present invention concerns a pharmaceutical composition comprising a therapeutically effective amount of a compound of formula I, and a pharmaceutically acceptable carrier. A therapeutically effective amount in this context is an amount sufficient to stabilize or to reduce viral infection, and in particular HCV infection, in infected subjects (e.g. humans). The “therapeutically effective amount” will vary depending on individual requirements in each particular case. Features that influence the dose are e.g. the severity of the disease to be treated, age, weight, general health condition etc. of the subject to be treated, route and form of administration.

In one aspect, the invention relates to the use of a compound of formula I, for the treatment of “treatment naive” patients, i.e. patients infected with HCV that are not previously treated against the infection.

In another aspect the invention relates to the use of a compound of formula I, the treatment of “treatment experienced” patients, i.e. patients infected with HCV that are previously treated against the infection and have subsequently relapsed.

In another aspect the invention relates to the use of a compound of formula I, the treatment of “non-responders”, i.e. patients infected with HCV that are previously treated but have failed to respond to the treatment.

In a further aspect, the present invention concerns a pharmaceutical composition comprising a prophylactically effective amount of a compound of formula I as specified herein, and a pharmaceutically acceptable carrier. A prophylactically effective amount in this context is an amount sufficient to act in a prophylactic way against HCV infection, in subjects being at risk of being infected.

In still a further aspect, this invention relates to a process of preparing a pharmaceutical composition as specified herein, which comprises intimately mixing a pharmaceutically acceptable carrier with a therapeutically or prophylactically effective amount of a compound of formula I, as specified herein.

Therefore, the compounds of the present invention may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in addition salt form or solvate, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, particularly, for administration orally, rectally, percutaneously, or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, lubricants, binders, disintegrating agents and the like in the case of powders, pills, capsules, and tablets. Because of their ease in administration, tablets and capsules represent the most advantageous oral dosage unit forms, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. The compounds of the present invention may also be administered via oral inhalation or insufflation in the form of a solution, a suspension or a dry powder using any art-known delivery system.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, suppositories, powder packets, wafers, injectable solutions or suspensions and the like, and segregated multiples thereof.

The compounds of formula I show activity against HCV and can be used in the treatment and/or prophylaxis of HCV infection or diseases associated with HCV. Typically the compounds of formula I can be used in the treatment of HCV infection or diseases associated with HCV. Diseases associated with HCV include progressive liver fibrosis, inflammation and necrosis leading to cirrhosis, end-stage liver disease, and HCC. A number of the compounds of this invention may be active against mutated strains of HCV. Additionally, many of the compounds of this invention may show a favourable pharmacokinetic profile and have attractive properties in terms of bioavailability, including an acceptable half-life, AUC (area under the curve) and peak values and lacking unfavourable phenomena such as insufficient quick onset and tissue retention.

The in vitro antiviral activity against HCV of the compounds of formula I can be tested in a cellular HCV replicon system based on Lohmann et al. (1999) Science 285:110-113, with the further modifications described by Krieger et al. (2001) Journal of Virology 75:4614-4624 (incorporated herein by reference), which is further exemplified in the examples section. This model, while not a complete infection model for HCV, is widely accepted as the most robust and efficient model of autonomous HCV RNA replication currently available. It will be appreciated that it is important to distinguish between compounds that specifically interfere with HCV functions from those that exert cytotoxic or cytostatic effects in the HCV replicon model, and as a consequence cause a decrease in HCV RNA or linked reporter enzyme concentration. Assays are known in the field for the evaluation of cellular cytotoxicity based for example on the activity of mitochondrial enzymes using fluorogenic redox dyes such as resazurin. Furthermore, cellular counter screens exist for the evaluation of non-selective inhibition of linked reporter gene activity, such as firefly luciferase. Appropriate cell types can be equipped by stable transfection with a luciferase reporter gene whose expression is dependent on a constitutively active gene promoter, and such cells can be used as a counter-screen to eliminate non-selective inhibitors.

Due to their antiviral properties, particularly their anti-HCV properties, the compounds of formula I, including any possible stereoisomers, the pharmaceutically acceptable addition salts or solvates thereof, are useful in the treatment of warm-blooded animals, in particular humans, infected with HCV. The compounds of formula I are further useful for the prophylaxis of HCV infections. The present invention furthermore relates to a method of treating a warm-blooded animal, in particular human, infected by HCV, or being at risk of infection by HCV, said method comprising the administration of an anti-HCV effective amount of a compound of formula I.

The compounds of the present invention may therefore be used as a medicine, in particular as an anti HCV medicine. Said use as a medicine or method of treatment comprises the systemic administration to HCV infected subjects or to subjects susceptible to HCV infection of an amount effective to combat the conditions associated with HCV infection.

The present invention also relates to the use of the present compounds in the manufacture of a medicament for the treatment or the prevention of HCV infection.

In a preferred embodiment, the present invention relates to the use of the compounds of formula I in the manufacture of a medicament for the treatment of HCV infection.

In general it is contemplated that an antiviral effective daily amount would be from about 0.01 to about 700 mg/kg, or about 0.5 to about 400 mg/kg, or about 1 to about 250 mg/kg, or about 2 to about 200 mg/kg, or about 10 to about 150 mg/kg body weight. It may be appropriate to administer the required dose as two, three, four or more sub-doses at appropriate intervals throughout the day. Said sub-doses may be formulated as unit dosage forms, for example, containing about 1 to about 5000 mg, or about 50 to about 3000 mg, or about 100 to about 1000 mg, or about 200 to about 600 mg, or about 100 to about 400 mg of active ingredient per unit dosage form.

The invention also relates to a combination of a compound of formula I, a pharmaceutically acceptable salt or solvate thereof, and another antiviral compound, in particular another anti-HCV compound. The term “combination” may relate to a product containing (a) a compound of formula I and (b) optionally another anti-HCV compound, as a combined preparation for simultaneous, separate or sequential use in treatment of HCV infections.

Anti-HCV compounds that can be used in such combinations include HCV polymerase inhibitors, HCV protease inhibitors, inhibitors of other targets in the HCV life cycle, and an immunomodulatory agents, and combinations thereof. HCV polymerase inhibitors include, NM283 (valopicitabine), R803, JTK-109, JTK-003, HCV-371, HCV-086, HCV-796 and R-1479, R-7128, MK-0608, VCH-759, PF-868554, GS9190, XTL-2125, NM-107, GSK625433, R-1626, BILB-1941, ANA-598, IDX-184, IDX-375, INX-189, MK-3281, MK-1220, ABT-333, PSI-7851, PSI-6130, GS-7977, VCH-916. Inhibitors of HCV proteases (NS2-NS3 inhibitors and NS3-NS4A inhibitors) include BILN-2061, VX-950 (telaprevir), GS-9132 (ACH-806), SCH-503034 (boceprevir), TMC435350 (also referred to as TMC435, Simeprevir), TMC493706, ITMN-191, MK-7009, BI-12202, BILN-2065, BI-201335, BMS-605339, R-7227, VX-500, BMS650032, VBY-376, VX-813, SCH-6, PHX-1766, ACH-1625, IDX-136, IDX-316. An example of an HCV NS5A inhibitor is BMS790052, A-831, A-689, NIM-811 and DEBIO-025 are examples of NS5B cyclophilin inhibitors.

Inhibitors of other targets in the HCV life cycle, including NS3 helicase; metalloprotease inhibitors; antisense oligonucleotide inhibitors, such as ISIS-14803 and AVI-4065; siRNA's such as SIRPLEX-140-N; vector-encoded short hairpin RNA (shRNA); DNAzymes; HCV specific ribozymes such as heptazyme, RPI.13919; entry inhibitors such as HepeX-C, HuMax-HepC; alpha glucosidase inhibitors such as celgosivir, UT-231B and the like; KPE-02003002; and BIVN 401.

Immunomodulatory agents include, natural and recombinant interferon isoform compounds, including α-interferon, β-interferon, γ-interferon, and ω-interferon, such as Intron A®, Roferon-A®, Canferon-A300®, Advaferon®, Infergen®, Humoferon®, Sumiferon MP®, Alfaferone®, IFN-beta®, and Feron®; polyethylene glycol derivatized (pegylated) interferon compounds, such as PEG interferon-α-2a (Pegasys®), PEG interferon-α-2b (PEG-Intron®), and pegylated IFN-α-con1; long acting formulations and derivatizations of interferon compounds such as the albumin-fused interferon albuferon α; compounds that stimulate the synthesis of interferon in cells, such as resiquimod; interleukins; compounds that enhance the development of type 1 helper T cell response, such as SCV-07; TOLL-like receptor agonists such as CpG-10101 (actilon), and isatoribine; thymosin α-1; ANA-245; ANA-246; histamine dihydrochloride; propagermanium; tetrachlorodecaoxide; ampligen; IMP-321; KRN-7000; antibodies, such as civacir and XTL-6865; and prophylactic and therapeutic vaccines such as InnoVac C and HCV E1E2/MF59.

Other antiviral agents include, ribavirin, amantadine, viramidine, nitazoxanide; telbivudine; NOV-205; taribavirin; inhibitors of internal ribosome entry; broad-spectrum viral inhibitors, such as IMPDH inhibitors, and mycophenolic acid and derivatives thereof, and including, but not limited to, VX-497 (merimepodib), VX-148, and/or VX-944); or combinations of any of the above.

Particular agents for use in said combinations include interferon-α (IFN-α), pegylated interferon-α or ribavirin, as well as therapeutics based on antibodies targeted against HCV epitopes, small interfering RNA (Si RNA), ribozymes, DNAzymes, antisense RNA, small molecule antagonists of for instance NS3 protease, NS3 helicase and NS5B polymerase.

In another aspect there are provided combinations of a compound of formula I as specified herein and an anti-HIV compound. The latter preferably are those HIV inhibitors that have a positive effect on drug metabolism and/or pharmacokinetics that improve bioavailability. An example of such an HIV inhibitor is ritonavir. As such, this invention further provides a combination comprising (a) a compound of formula I or a pharmaceutically acceptable salt or solvate thereof; and (b) ritonavir or a pharmaceutically acceptable salt thereof. The compound ritonavir, its pharmaceutically acceptable salts, and methods for its preparation are described in WO 94/14436. U.S. Pat. No. 6,037,157, and references cited therein: U.S. Pat. No. 5,484,801, U.S. Ser. No. 08/402,690, WO 95/07696, and WO 95/09614, disclose preferred dosage forms of ritonavir.

The invention also concerns a process for preparing a combination as described herein, comprising the step of combining a compound of formula I and another agent, such as an antiviral, including an anti-HCV or anti-HIV agent, in particular those mentioned above.

The said combinations may find use in the manufacture of a medicament for treating HCV infection in a mammal infected therewith, said combination in particular comprising a compound of formula I, as specified above and interferon-α (IFN-α), pegylated interferon-α, or ribavirin. Or the invention provides a method of treating a mammal, in particular a human, infected with HCV comprising the administration to said mammal of an effective amount of a combination as specified herein. In particular, said treating comprises the systemic administration of the said combination, and an effective amount is such amount that is effective in treating the clinical conditions associated with HCV infection.

In one embodiment the above-mentioned combinations are formulated in the form of a pharmaceutical composition that includes the active ingredients described above and a carrier, as described above. Each of the active ingredients may be formulated separately and the formulations may be co-administered, or one formulation containing both and if desired further active ingredients may be provided. In the former instance, the combinations may also be formulated as a combined preparation for simultaneous, separate or sequential use in HCV therapy. The said composition may take any of the forms described above. In one embodiment, both ingredients are formulated in one dosage form such as a fixed dosage combination. In a particular embodiment, the present invention provides a pharmaceutical composition comprising (a) a therapeutically effective amount of a compound of formula I, including a possible stereoisomeric form thereof, or a pharmaceutically acceptable salt thereof, or a pharmaceutically acceptable solvate thereof, and (b) a therapeutically effective amount of ritonavir or a pharmaceutically acceptable salt thereof, and (c) a carrier.

The individual components of the combinations of the present invention can be administered separately at different times during the course of therapy or concurrently in divided or single combination forms. The present invention is meant to embrace all such regimes of simultaneous or alternating treatment and the term “administering” is to be interpreted accordingly. In a preferred embodiment, the separate dosage forms are administered simultaneously.

In one embodiment, the combinations of the present invention contain an amount of ritonavir, or a pharmaceutically acceptable salt thereof, that is sufficient to clinically improve the bioavailability of the compound of formula I relative to the bioavailability when said compound of formula I is administered alone. Or, the combinations of the present invention contains an amount of ritonavir, or a pharmaceutically acceptable salt thereof, which is sufficient to increase at least one of the pharmacokinetic variables of the compound of formula I selected from t_{1/2}, Cₘᵢₙ, Cₘₐₓ, Cₛₛ, AUC at 12 hours, or AUC at 24 hours, relative to said at least one pharmacokinetic variable when the compound of formula I is administered alone.

The combinations of this invention can be administered to humans in dosage ranges specific for each component comprised in said combinations, e.g. the compound of formula I as specified above, and ritonavir or a pharmaceutically acceptable salt, may have dosage levels in the range of 0.02 to 5.0 g/day.

The weight ratio of the compound of formula I to ritonavir may be in the range of from about 30:1 to about 1:15, or about 15:1 to about 1:10, or about 15:1 to about 1:1, or about 10:1 to about 1:1, or about 8:1 to about 1:1, or about 5:1 to about 1:1, or about 3:1 to about 1:1, or about 2:1 to 1:1. The compound formula I and ritonavir may be co-administered once or twice a day, preferably orally, wherein the amount of the compound of formula I per dose is as described above; and the amount of ritonavir per dose is from 1 to about 2500 mg, or about 50 to about 1500 mg, or about 100 to about 800 mg, or about 100 to about 400 mg, or 40 to about 100 mg of ritonavir.

### DETAILED DESCRIPTION OF THE EMBODIMENTS

Various embodiments of the invention and intermediates therefore will now be illustrated by the following examples. The Examples are just intended to further illustrate the invention and are by no means limiting the scope of the invention. The compound names were generated by ChemDraw Ultra software, Cambridgesoft, version 12.0.2.

### Intermediate 1

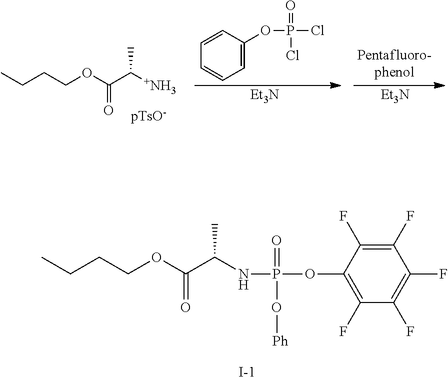

### (2S)-butyl 2-(((perfluorophenoxy)(phenoxy)phosphoryl)amino)propanoate (I-1)

Phenyl dichlorophosphate (12.4 ml, 83.1 mmol) was added to a cooled (−20° C.) slurry of the pTs salt of (S)-butyl 2-aminopropanoate (26.4 g, 83.1 mmol) in dichloromethane (DCM) (200 ml). The mixture was stirred for 10 min then triethylamine (25.5 ml, 183 mmol) was added drop wise during 15 min. The mixture was stirred at −20° C. for 1 h then at 0° C. for 30 min. The mixture was kept cooled in an ice-bath and pentafluorophenol (15.3 g, 0.08 mol) was added followed by a drop wise addition of triethylamine (11.6 ml, 0.08 mol). The mixture was stirred overnight and then left to attain 20° C. Diethyl ether was added and the mixture was filtered through Celite, concentrated and purified by column chromatography on silica eluted with p. ether/EtOAc (9:1→8:2). Fractions containing product were pooled, concentrated and crystallized from p. ether EtOAc (9:1) which gave the title compound (2.23 g) as one diastereomer. The mother liquor was concentrated and crystallised by addition of p. ether which gave further title product (1.92 g) as a crystalline mixture of diastereomers. Purification of the mother liquor by column chromatography on silica gel eluted with p. ether/EtOAc (9:1→8:2) gave further 1.52 g of the title compound.

### Intermediate 2

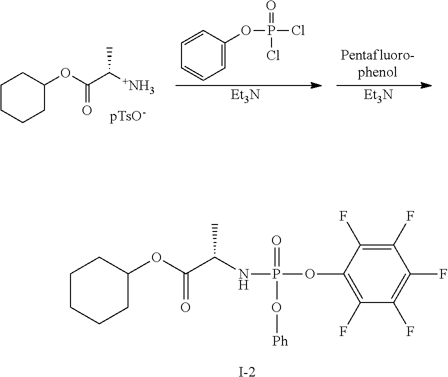

### (2S)-cyclohexyl 2-(((perfluorophenoxy)(phenoxy)phosphoryl)amino)propanoate (I-2)

L-alanine cyclohexyl ester (22 g, 64 mmol) was reacted according to the method described for the preparation of Intermediate 1, which gave the title compound (32 g, 28%).

### Intermediate 3

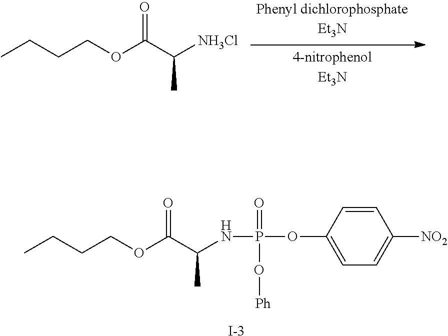

### (2S)-butyl 2-(((4-nitrophenoxy)(phenoxy)phosphoryl)amino)propanoate (I-3)

Phenyl dichlorophosphate (7.4 mL, 49.5 mmol) was added at −30° C. under argon in one portion to a solution of the hydrochloride of (S)-butyl 2-aminopropanoate (9.0 g, 49.5 mmol) in CH₂Cl₂ (100 mL). After 10 min triethylamine (15 ml, 109 mmol) was added dropwise and the reaction mixture was allowed to attain room temperature and was stirred for 5 h under Ar. The reaction mixture was then cooled on an ice-bath and 4-nitrophenol (6.9 g, 49.5 mmol) was added in one portion followed by dropwise addition of triethylamine (6.9 mL, 49.5 mmol) under Ar. The reaction mixture was allowed to reach room temperature, stirred under Ar for 72 h and then concentrated. The residue was dissolved in THF (300 mL) and the white precipitate formed was filtered off and washed several times with THF. The filtrate was concentrated and the afforded crude product was purified by column chromatography (n-hexane/EtOAc (92:8)-(34:66)) which gave the title compound (16.4 g, 78.6%).

### Intermediate 4

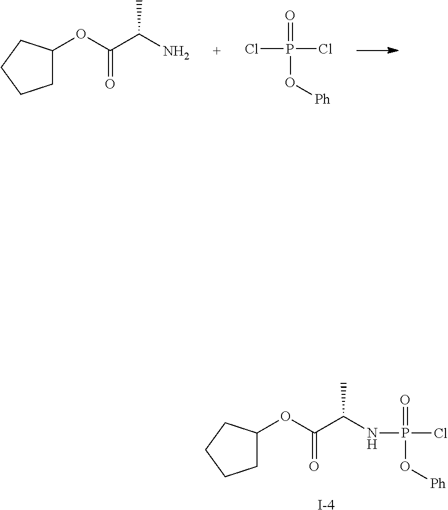

### (2S)-cyclopentyl 2-((chloro(phenoxy)phosphoryl)amino)propanoate (I-4)

Triethylamine (1.39 ml, 10 mmol) was added slowly at approximately −25 to a solution of (S)-cyclopentyl 2-aminopropanoate (1.65 g, 5 mmol) and phenyl dichlorophosphate (1.05 g, 5 mmol) in DCM (15 ml). After 90 min Et₃N (1.39 mL, 10 mmol) was added slowly and on completion of addition, the reaction mixture was allowed to attain room temperature and stirred overnight and the concentrated. The afforded residue was purified by column chromatography on silica eluted with EtOAc/i-hexane: 25/75, which gave the title compound (1.61 g, 56%).

### Intermediate 5

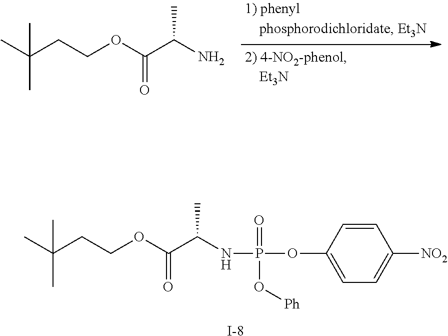

### (2S)-3,3-dimethylbutyl 2-(((4-nitrophenoxy)(phenoxy)phosphoryl)amino)propanoate (I-5)

Phenyl dichlorophosphate (3.2 g, 15 mmol) was added under nitrogen at −30° C. to a solution of (S)-3,3-dimethylbutyl 2-aminopropanoate (5.2 g, 15 mmol) in DCM (80 ml), followed by dropwise addition of triethylamine (3.0 mg, 30 mmol). The mixture was allowed to attain room temperature and stirred overnight, then cooled to about 5° C. and 4-nitrophenol (2.1 g, 15 mmol) was added as a solid followed by dropwise addition of triethylamine (1.5 g, 15 mmol) and the mixture was stirred for 4 hours at room temperature, then concentrated under reduced pressure, diluted with ethyl acetate (50 ml) and ether (50 ml) and left at room temperature overnight. The triethylamine-HCl salt was filtered of and the filtrate was concentrated under reduced pressure. The afforded residue was purified by chromatography on silica gel eluted with isohexane/EtOAc, which gave the title compound (5.8 g, 86%).

### Intermediate 6

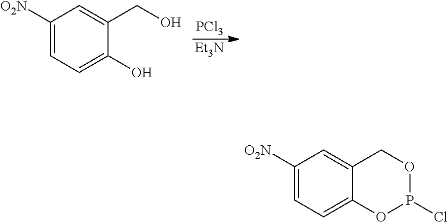

### 2-Chloro-6-nitro-4H-benzo[d][1,3,2]dioxaphosphinine (I-6)

Phosphorous trichloride (2 mmol) was slowly added to a suspension of 2-hydroxy-5-nitrobenzyl alcohol (2 mmol) in dry ether (10 mL), at −20° C. under nitrogen. After 10 min a solution of triethylamine (4.2 mmol) in dry ether (10 mL) was added over a period of 45 min keeping the internal temperature at −20° C. The mixture was stirred at −20° C. for 15 min and then at room temperature for 1 h 30 min, then diluted with dry ether (10 mL) and filtered through a pad of dry Celite® under nitrogen. The solvent was removed under vacuum which gave the P-reagent (249 mg, 53%) as a white solid which was used in the phosphorylation step without further purification.

### Intermediate 7

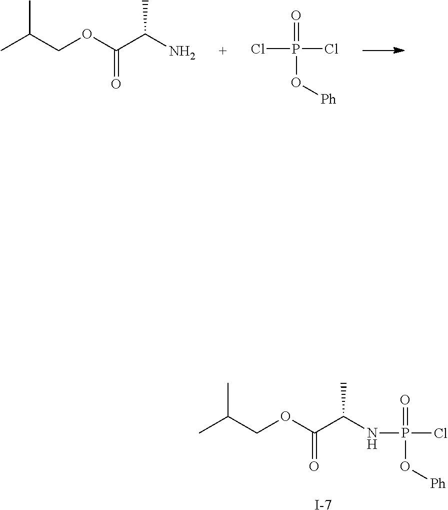

### (2S)-cyclopentyl 2-((chloro(phenoxy)phosphoryl)amino)propanoate (I-7)

The HCl salt of L-alanine isobutyl ester (2.27 g, 12.5 mmol) was reacted according to the method described for the preparation of Intermediate 4, which gave the title compound (1.21 g, 36%).

### Intermediate 8

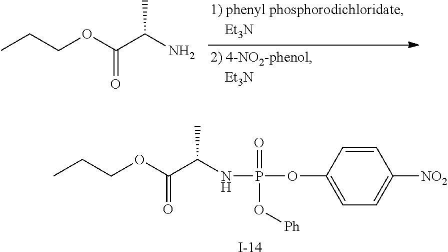

### (2S)-propyl 2-(((4-nitrophenoxy)(phenoxy)phosphoryl)amino)propanoate (I-8)

The procedure described for the preparation of I-5 was followed but using (S)-propyl 2-aminopropanoate (4.44 g, 26.5 mmol) instead of (S)-3,3-dimethylbutyl 2-aminopropanoate, which gave the title compound (6.9 g, 64%).

### Intermediate 9

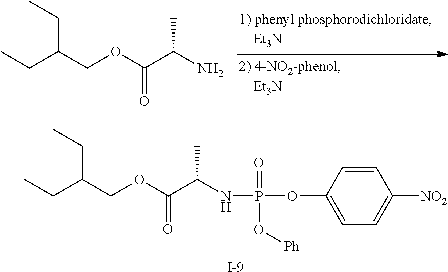

### (2S)-2-Ethylbutyl 2-(((4-nitrophenoxy)(phenoxy)phosphoryl)amino)propanoate (I-9)

The procedure described for the preparation of I-5 was followed but using (S)-2-ethylbutyl 2-aminopropanoate (5.2 g, 15 mmol) instead of (S)-3,3-dimethylbutyl 2-aminopropanoate, which gave the title compound (6.8 g, 84%).

### Intermediate 10

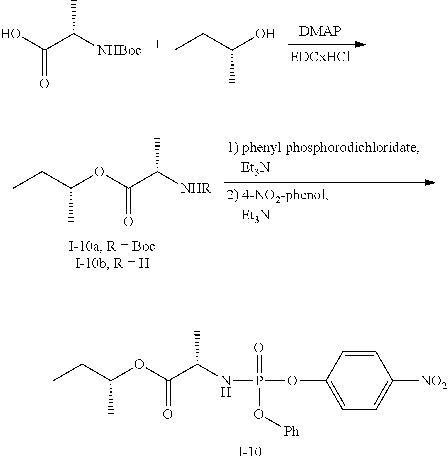

### Step a) (S)—(R)-sec-butyl 2-((tert-butoxycarbonyl)amino)propanoate (I-10a)

L-Boc-Alanine (2.18 g, 11.5 mmol) was dissolved in dry DCM (40 mL) and (R)-butan-2-ol (938 mg, 12.6 mmol) was added. The mixture was cooled to about 5° C. and EDC (3.31 g, 17.2 mmol) was added in one portion followed by portionwise addition of DMAP (140 mg, 1.15 mmol). The mixture was allowed to attain room temperature and stirred overnight, then diluted with ethyl acetate (˜300 ml) and the organic phase was washed three times with a saturated solution of sodium hydrogen carbonate and once with brine. The organic phase was dried over sodium sulphate and concentrated under reduced pressure. The product was isolated by silica gel chromatography eluted with isohexane and 10% EtOAc, which gave the title compound (2.78 g, 98%).

### Step b) (S)—(R)-Sec-butyl 2-aminopropanoate (I-10b)

A mixture of I-10a (2.77 g, 11.3 mmol) and p-toluene sulphonic acid mono hydrate (2.15 g, 11.3 mmol) was stirred for 16 h at 65° C., then concentrated under reduced pressure. The afforded residue was crystallised from diethyl ether, which gave the title compound (3.20 g, 89%).

### (2S)—(R)-Sec-butyl 2-(((4-nitrophenoxy)(phenoxy)phosphoryl)amino)propanoate (I-10)

The procedure described for the preparation of I-5 was followed but using (S)—(R)-sec-butyl 2-aminopropanoate (3.15 g, 9.92 mmol) instead of (S)-3,3-dimethylbutyl 2-aminopropanoate, which gave the title compound (4.19 g, 79%).

### Intermediate 11

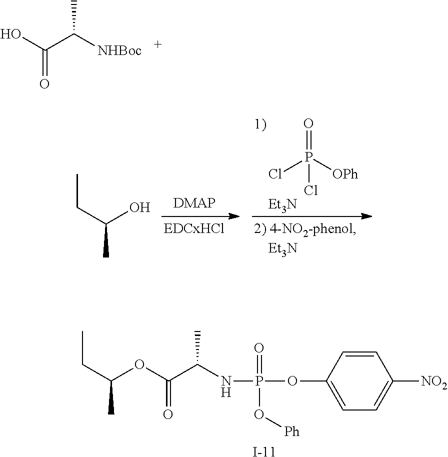

### (2S)—(S)-Sec-butyl 2-(((4-nitrophenoxy)(phenoxy)phosphoryl)amino)propanoate (I-11)

The procedure described for the preparation of I-10 was followed but using (S)-butan-2-ol instead of (R)-butan-2-ol, which gave the title compound in 91% yield.

### Intermediate 12

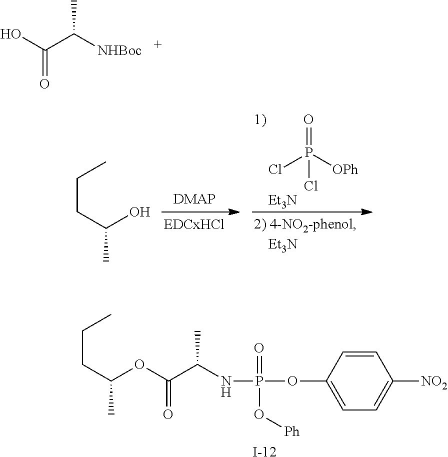

### ((2S)—(R)-Pentan-2-yl 2-(((4-nitrophenoxy)(phenoxy)phosphoryl)amino)propanoate (I-12)

The procedure described for the preparation of I-10 was followed but using (R)-pentan-2-ol instead of (R)-butan-2-ol, which gave the title compound (4.6 g).

### Intermediate 13

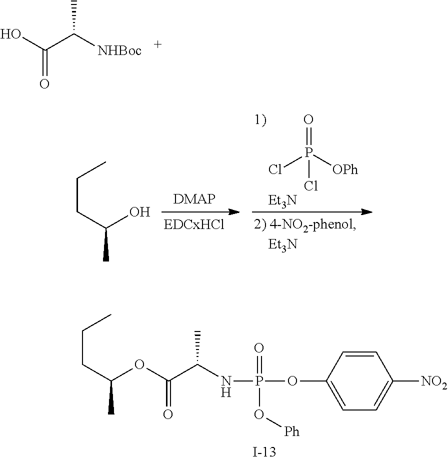

### (2S)—(S)-Pentan-2-yl 2-(((4-nitrophenoxy)(phenoxy)phosphoryl)amino)propanoate (I-13)

The procedure described for the preparation of I-10 was followed but using (S)-pentan-2-ol instead of (R)-butan-2-ol, which gave the title compound (8.3 g).

### Intermediate 14, Large Scale Preparation of I-9

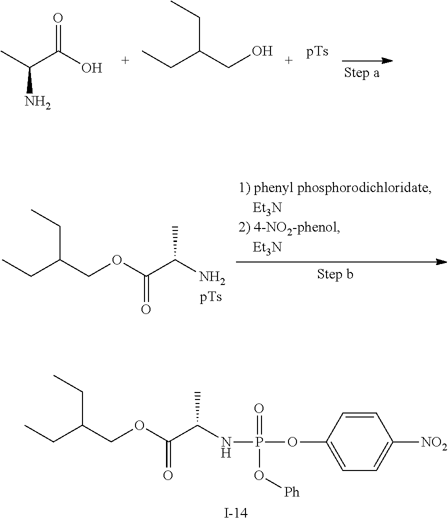

### Step a) (S)-2-ethylbutyl 2-aminopropanoate

A slurry of L-alanine (27.2 g, 305 mmol), p-toluenesulfonic acid xH₂O (58.0 g, 305 mmol) and 2-ethyl-1-butanol (75 ml, 610 mmol) in toluene (700 ml) was heated to reflux in a 1 l flask equipped with a dean-stark trap and refluxed for 14 h. The reaction mixture was filtered and the filtrate concentrated. The afforded residue was dissolved in Et₂O (250 ml) and the solution was seeded with a previously formed intermediate pTs-ammonium salt, whereby crystals slowly precipitated. After 3 h, when a thick slurry of crystals was formed, isohexane (250 ml) was added and the flask was placed at 5° C. for 2 h, then at −20° C. for 2 h 30 min. The slurry is filtered, and the crystals were washed with cold Et₂O/iso-hexane 1/4 and dried in vacuum overnight. Yield 95.5 g, 91%.

### Step b) (2S)-2-Ethylbutyl 2-(((4-nitrophenoxy)(phenoxy)phosphoryl)amino)propanoate

To a 2 l three-necked reaction flask fitted with a mechanical stirrer was added phenyl dichlorophosphate (41.2 ml, 276.4 mmol) and CH₂Cl₂ (300 ml). The solution was cooled to ˜0° C. with ice/H₂O under N₂-atmosphere. The amine from step a (95.5 g, 276.4 mmol) and CH₂Cl₂ (100 ml) was added and the slurry was cooled with ice/H₂O/NaCl keeping the inner temperature of the flask was to ˜−12° C. After 45 min, triethylamine (848 ml, 608.2 mmol) in CH₂Cl₂ (200 ml) was added slowly over 65 minutes. After the addition the temperature is slowly raised to 20° C. After 3 h, the reaction mixture was cooled to ˜0° C. with ice/H₂O and 4-nitrophenol (38.5 g, 276.4 mmol) was added in one portion followed by a dropwise addition of triethylamine (38.5 ml, 276.4 mmol) in CH₂Cl₂ (150 ml) over ˜60 min. The reaction mixture is left stirring overnight, then filtered, washed with iso-hexane and concentrated. THF (350 ml) was added to the residue and the slurry was stirred at room temperature for ˜1.5 h, then put in at ˜5° C. for ˜1 h and filtered. The precipitate was filtered out and washed with THF/iso-hexane 50/50. The filtrate is evaporated which gave 200 g of a viscous syrup.

The syrup was purified by flash chromatography using YMC-gel, column size 90×150 mm. Eluent: EtOAc/i-hexane: 25/75-35/65, which gave 95.3 gr of yellowish syrup, the afforded syrup was subjected to further purification by flash chromatography on silica, which gave the title compound (77.0 g, 62%)

### Crystallization

10 g of the above compound was dissolved in diisopropyl ether (˜20 ml), iso-hexane (˜15 ml) was added slowly until permanent weak cloudiness appeared. The solution was warmed until the solution became clear. The formed 2-phase system was seeded with crystals from previously prepared I-9 and a thick precipitation of crystals was formed. The slurry was stirred at room temperature for 3 h, the filtered and the solid washed with diisopropyl ether/isohexane 50:50 (2×20 ml), which gave a first crop of crystals (2.473 g).

The filtrate was put at ˜5° C. overnight and more crystals precipitated. The slurry was filtered and washed with isohexane (2×10 ml)

which gave a 2:nd crop of crystals (1.031 g), total yield: 3.50 g, 35%.

The mother liquor was concentrated and subjected to further purification by crystallisation. (2S)-2-Ethylbutyl 2-(((4-nitrophenoxy)(phenoxy)phosphoryl)amino)propanoate (58.24 g) was dissolved in diisopropyl ether (500 mL) and washed with aqueous saturated potassium hydrogen carbonate (4×120 mL). The organic phase was dried with sodium sulphate, filtered and concentrated. The afforded oil (56.7 g) was suspended in a mixture of diethyl ether and n-heptane (1:1; 100 mL) and the mixture was concentrated under reduced pressure. The procedure was repeated twice which gave a white solid. The solid was suspended in a mixture of 45% diethyl ether in n-hexane (700 mL) and mixture was heated to 35° C. to give homogeneous mixture, then cooled to −10° C. during which crystallization occurred. The mixture was stirred for 1 h at −10° C. and then at −44° C. for 5 hours. The mixture was filtered and washed with a cold mixture (−44° C.) of 45% diethyl ether in n-hexane (200 mL) which gave the product as a white solid (25 g; 44%). The product was analyzed by ¹H NMR and ³¹P NMR which showed that product was obtained in 96% diastereomeric excess (98:2 diastereomeric ratio).

### Example 1

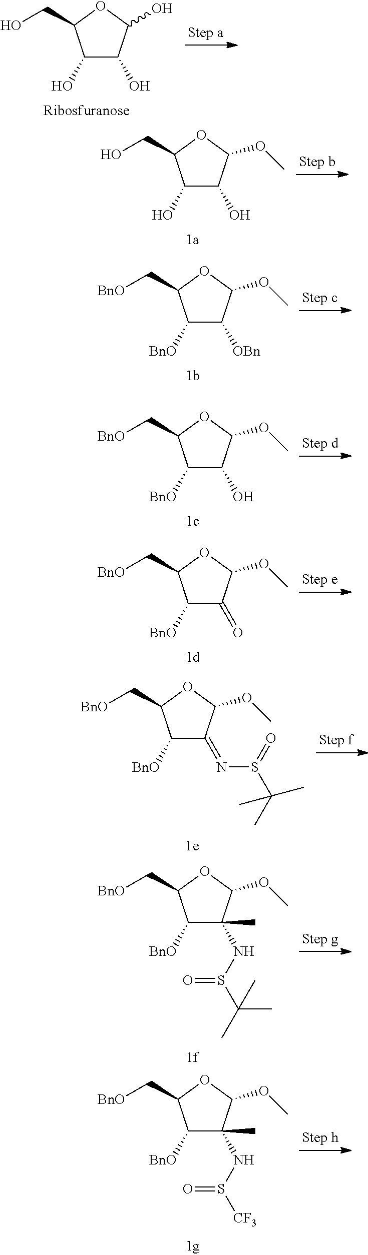

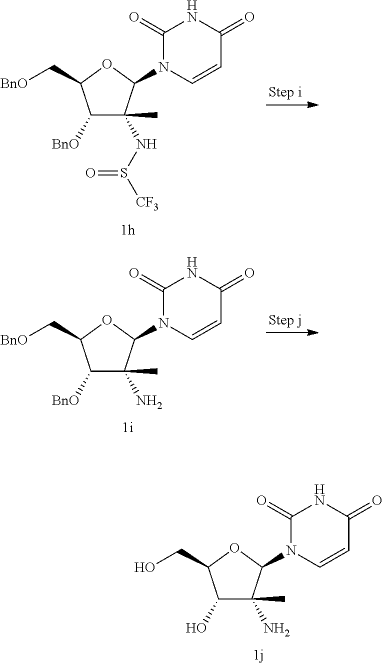 [SMILES: C[C@@]1(NS(=O)C(F)(F)F)[C@H](OCc2ccccc2)[C@@H](COCc2ccccc2)O[C@H]1n1ccc(=O)[nH]c1=O ; C[C@@]1(N)[C@H](OCc2ccccc2)[C@@H](COCc2ccccc2)O[C@H]1n1ccc(=O)[nH]c1=O ; C[C@@]1(N)[C@H](O)[C@@H](CO)O[C@H]1n1ccc(=O)[nH]c1=O]

### Step a) (2R,3S,4R,5S)-2-(Hydroxymethyl)-5-methoxytetrahydrofuran-3,4-diol (1a)

Cone. H₂SO₄ (6 mL) was added dropwise at 0° C. to a solution of D-Ribose (100 g, 133.2 mmol) in MeOH (700 mL) and the same temperature was kept for 24 h. After completion of the reaction, the reaction mixture was neutralized with Amberlyst A-26 (OH⁻) ion exchange resin. The solvent was removed under reduced pressure which gave the title compound (100 g, 91%) as a yellow liquid which was sufficiently pure and used in the next step without further purification.

### Step b) (2R,3R,4R,5S)-3,4-Bis(benzyloxy)-2-((benzyloxy)methyl)-5-methoxytetrahydrofuran (1b)

To a solution of 1a (100 g, 609.7 mmol) in DMF (1.5 L), sodium hydride (150 g, 3.71 mol) was added in portions at 0° C. and allowed to stir at same temperature for 45 min. Benzyl bromide (450 mL, 3.71 mol) was added dropwise at 0° C. and the reaction mixture was stirred at room temperature for 16 h. After completion of the reaction (TLC), the reaction mixture was quenched with ice-cold water (1 L), and the compound was extracted with EtOAc (2 L), washed with water (500 mL) and dried over sodium sulphate. After removal of the solvent the crude was purified by column chromatography on silica gel (15-30% EtOAc in p. ether) which gave the title compound (240 g, 90%).

### Step c) (2S,3R,4S,5R)-4-(Benzyloxy)-5-((benzyloxy)methyl)-2-methoxytetrahydrofuran-3-ol (1c)

To a solution of 1b (180 g, 414.7 mmol) in DCM (1.8 L), SnCl₄ (450 mL, 414.3 mmol) was added drop wise at 0° C. and the reaction mixture was stirred at 0° C. for 24 h. After completion of reaction (TLC), the reaction mixture was quenched with water (1 L), and the compound was extracted with EtOAc (2 L). The organic layer was washed with 10% aqueous NaHCO₃ solution (200 mL) and 0.5 N HCl solutions (200 mL) followed by drying over sodium sulphate. After removal of the solvent the crude was purified by column chromatography on silica gel (15% EtOAc in p. ether) which gave the title compound (120 g, 86%).

### Step d) (2S,4R,5R)-4-(Benzyloxy)-5-((benzyloxy)methyl)-2-methoxydihydrofuran-3(2H)-one (1d)

To a mixture of Dess-Martin periodinane (105 g, 247.6 mmol) in DCM (250 mL), a degasified solution of compound 1c (50 g, 145.3 mmol) in DCM (250 mL) was added drop wise at 0° C. and the reaction mixture was stirred at room temperature for 24 h. After completion of the reaction (TLC), the solvent was concentrated under reduced pressure at room temperature (30° C.) and to the residue was added diethyl ether (2 L) and stirred for 15 min. After filtration the filtrate was washed with saturated aqueous sodium thiosulphate solution (400 mL) and 10% aqueous NaHCO₃ solution (400 mL) followed by drying over sodium sulphate. The solvent was removed under reduced pressure which gave the title compound (43 g, 86%) sufficiently pure to be used in the next step without further purification.

### Step e) (Z)—N-((2S,4S,5R)-4-(Benzyloxy)-5-((benzyloxy)methyl)-2-methoxydihydrofuran-3(2H)-ylidene)-2-methylpropane-2-sulfinamide (1e)

To a solution of compound 1d (15 g, 43.8 mmol) in THF (75 mL), titanium tetraethoxide (16 mL, 74.5 mmol) in THF (75 mL) was added dropwise at room temperature and then 2-methyl-2-propane sulfinamide (6 g, 43.8 mmol) was added rapidly and the reaction mixture was heated to 60° C. for 6 h. After completion of the reaction (TLC), the reaction mixture was poured in to a saturated sodium chloride solution (100 mL), filtered through a Celite bed, and the filtrate was extracted with EtOAc (500 mL), washed with water (100 mL), dried over sodium sulphate. After removal of the solvent the crude was purified by column chromatography on silica gel (7% EtOAc in p. ether) which gave the title compound (11 g, 58%).

### Step f) N-((2S,3R,4S,5R)-4-(Benzyloxy)-5-((benzyloxy)methyl)-2-methoxy-3-methyltetrahydrofuran-3-yl)-2-methylpropane-2-sulfinamide (1f)

To a solution of compound 1e (16 g, 35.24 mmol) in dry THF (160 mL), methyl lithium (50 mL, 1.4 M in THF, 70.48 mmol) was added dropwise at −78° C. and reaction mixture was stirred at the same temperature for 30 min. After completion of the reaction (TLC), it was quenched with saturated ammonium chloride solution (25 mL) and compound was extracted with EtOAc (200 mL), washed with water (50 mL) and dried over sodium sulphate. After removal of the solvent the crude was purified by column chromatography on silica gel (230-400 mesh, 18-25% EtOAc in p. ether) which gave a diastereomeric mixture of the title compound (6 g, 34%).

### Step q) N-((2S,3R,4S,5R)-4-(Benzyloxy)-5-((benzyloxy)methyl)-2-methoxy-3-methyltetrahydrofuran-3-yl)-2,2,2-trifluoroacetamide (1g)

To a solution of compound 1f (6.1 g, 13.2 mmol) in 1,4-dioxane (6 mL), 3M HCl in dioxane (6.6 mL, 19.8 mmol) was added at 0° C. and warmed to room temperature and stirred for 1 h. After completion of the reaction (TLC), the solvent was removed under reduced pressure to give the title compound which was used in the next step without further purification.

To a solution of the afforded 2′-amino compound in DCM (60 mL), pyridine (2.7 mL, 31.7 mmol) and trifluoroacetic anhydride (4.1 mL, 27.7 mmol) were added at −40° C. and slowly warmed to room temperature for 3 h. The reaction mixture was diluted with DCM (100 mL), washed with water (100 mL), dried over sodium sulphate. After removal of the solvent the crude was purified by column chromatography on silica gel (10-12% EtOAc in p. ether) which gave the title compound (3.6 g, 60%).

### Step h) N-((2R,3R,4S,5R)-4-(Benzyloxy)-5-((benzyloxy)methyl)-2-(2,4-dioxo-3,4-dihydropyrimidin-1 (2H)-yl)-3-methyltetrahydrofuran-3-yl)-2,2,2-trifluoroacetamide (1 h)

A mixture of uracil (0.49 g, 4.41 mmol), N,O-bis(trimethylsilylacetamide) (2.26 mL, 9.26 mmol) and dry acetonitrile (15 mL) were heated at 85° C. for 30 min. The clear solution formed above was cooled to room temperature and a solution of compound 1g (1 g, 2.2 mmol) in dry acetonitrile (10 mL) was added dropwise and at 0° C. Then trimethylsilyl trifluoromethanesulphonate (0.6 mL, 3.3 mmol) was added and the reaction mixture was heated to 80° C. for 5 h. An additional lot of trimethylsilyl trifluoromethanesulphonate (0.6 mL, 3.3 mmol) was added and heating was continued for 16 h. After completion of the reaction (TLC), the solvent was removed under reduced pressure and the residue was taken in EtOAc (50 mL), washed with 10% aqueous NaHCO₃ solution (25 mL) and dried over sodium sulphate. After removal of the solvent the crude was purified by column chromatography on silica gel (230-400 mesh, 30% EtOAc in p. ether) which gave the title compound (0.25 g, 21%).

### Step i) 1-((2R,3R,4S,5R)-3-amino-4-(benzyloxy)-5-((benzyloxy)methyl)-3-methyltetrahydrofuran-2-yl)pyrimidine-2,4(1H,3H)-dione (1i)

A mixture of compound 1h (1 g, 1.8 mmol), NaOH (0.2 g, 5.6 mmol), 10% aqueous solution of Na₂CO₃ (5 mL) and MeOH (10 mL) was heated at 80° C. for 48 h. After completion of the reaction (TLC), the MeOH was removed under reduced pressure and the residue was taken in EtOAc (50 mL), washed with water (10 mL) and dried over sodium sulphate. The solvent was removed under reduced pressure which gave the title compound (0.6 g, 74%) which was used in next step without further purification.

### Step j) 1-((2R,3R,4S,5R)-3-amino-4-hydroxy-5-(hydroxymethyl)-3-methyltetrahydrofuran-2-yl)pyrimidine-2,4(1H,3H)-dione (1j)

The mixture of compound 1i (1 g, 2.2 mmol), 3N HCl in MeOH (10 mL), 20% Pd(OH)₂/C (0.4 g) was hydrogenated at room temperature for 6 h. After completion of the reaction (TLC), the reaction mixture was filtered, basified with ammonia, concentrated and the crude purified on silica gel (230-400 mesh, 10% MeOH in chloroform) which gave the title compound (0.4 g, 68%).

### Example 2

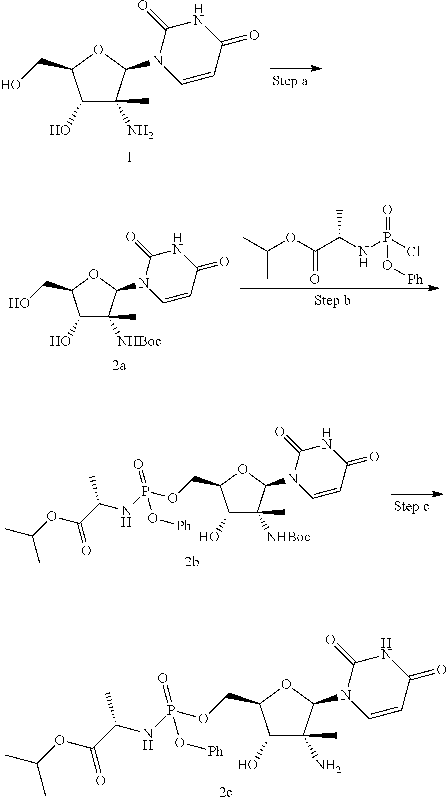

### Step a) tert-Butyl((2R,3R,4S,5R)-2-(2,4-dioxo-3,4-dihydropyrimidin-1(2H)-yl)-4-hydroxy-5-(hydroxymethyl)-3-methyltetrahydrofuran-3-yl)carbamate (2a)

The uracil-nucleoside 1 (291 mg, 1.13 mmol) was dissolved in acetonitrile/water:1/1 (3 mL) and triethylamine (2.50 mmol) was added. To the stirred solution was then added di-tert-butyl dicarbonate in portions of 1.13 mmol every 5 hours. Upon completion (2 to 3 days) the mixture was evaporated onto silica-gel and the residue purified by flash chromatography using gradient DCM/MeOH:98/2 to 92/8, which gave the title compound (324 mg, 80%). MS: 358.3 [M+H].

### Step b) (2S)-isopropyl 2-(((((2R,3S,4R,5R)-4-((tert-butoxycarbonyl)amino)-5-(2,4-dioxo-3,4-dihydropyrimidin-1(2H)-yl)-3-hydroxy-4-methyltetrahydrofuran-2-yl)methoxy)(phenoxy)-phosphoryl)amino)propanoate (2b)

N-methylimidazole (3.60 mmol) was added under nitrogen to a solution of the protected nucleoside 2a (0.90 mmol) in dry DCM (10 mL). The solution was cooled to −10° C. and a solution of aryl phosphoramidate reagent (1.1 mmol) in DCM (3 mL) was added. The cooling was removed and the reaction was stirred at room temperature. After 2 h, more aryl phosphoramidate reagent (0.70 mmol) was added and the reaction was stored at +4° C. overnight. The reaction was then quenched by addition of methanol and concentrated to dryness. The residue was purified by flash chromatography using gradient DCM/MeOH:100/0 to 96/4 which gave the title compound (299 mg, 53%) as a mixture of phosphorous diastereomers. MS: 627.5 [M+H].

### Step c) (2S)-isopropyl 2-(((((2R,3S,4R,5R)-4-amino-5-(2,4-dioxo-3,4-dihydropyrimidin-1(2H)-yl)-3-hydroxy-4-methyltetrahydrofuran-2-yl)methoxy)(phenoxy)phosphoryl)amino)propanoate (2c)

Compound 2b (0.48 mmol) was taken into 60% acetic acid (10 mL) and heated at 90 for 8 h, whereafter the solvent was removed and the crude residue purified by reverse-phase HPLC using a gradient of acetonitrile/water buffered with 10 mM ammonium acetate which gave the title compound (131 mg, 52%) as a mixture of phosphorous diastereomers. MS: 527.4 [M+H].

### Example 3

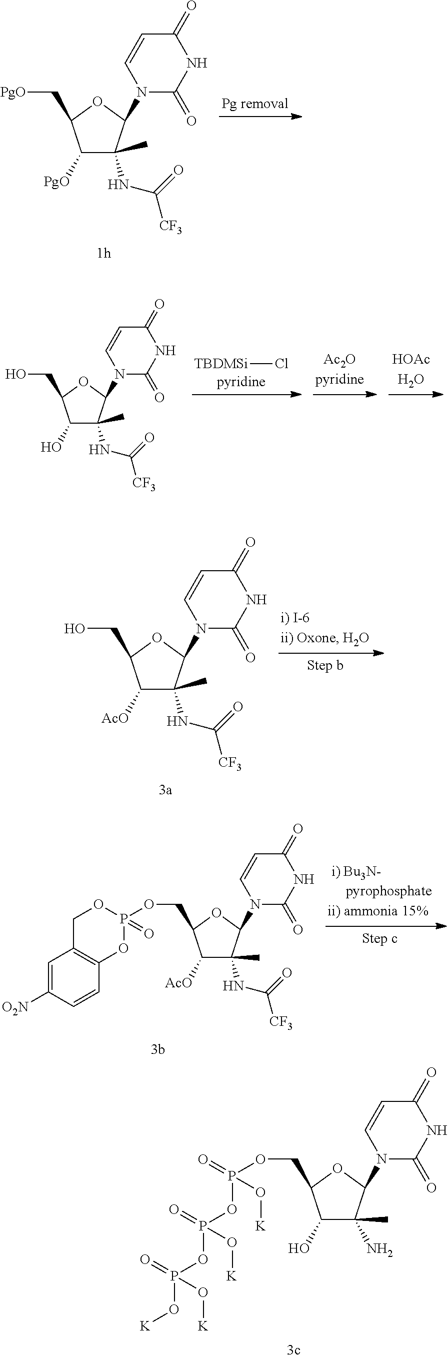

### Step a) (2R,3S,4R,5R)-5-(2,4-dioxo-3,4-dihydropyrimidin-1(2H)-yl)-2-(hydroxymethyl)-4-methyl-4-(2,2,2-trifluoroacetamido)tetrahydrofuran-3-yl acetate (3a)

The title compound was achieved subjecting compound 1h to the sequence debenzylation, tritylation, acetylation and finally detritylation using standard conditions.

### Step b) (2R,3S,4R,5R)-5-(2,4-dioxo-3,4-dihydropyrimidin-1(2H)-yl)-4-methyl-2-(((6-nitro-2-oxido-4H-benzo[d][1,3,2]dioxaphosphinin-2-yl)oxy)methyl)-4-(2,2,2-trifluoroacetamido)tetrahydrofuran-3-yl acetate (3b)

The 5′-O-unprotected nucleoside 2a (0.15 mmol) was dissolved in a mixture of acetonitrile/DCM:2/1 (4 mL) and the solution cooled to −20° C. under nitrogen. To the solution was added triethylamine (0.33 mmol) followed by 5-nitrocyclosalgenylchlorophosphite prepared above (0.30 mmol) as a solution in DCM (1 mL). The cooling bath was removed and the reaction stirred at room temperature for 1 h 30 min. After this time the reaction was cooled to −5° C. and a solution of Oxone® (0.60 mmol) in water (3 mL) was added and the two-phase system vigorously stirred for 15 min. The mixture was diluted with ethyl acetate (20 mL), the phases were separated and the organic phase washed with cold water (2×5 mL) and then dried over sodium sulphate. EtOAc was removed by evaporation and the residue taken into DCM (10 mL) and filtered again. Removable of the solvent afforded the desired intermediate 66 mg (72%) that was taken into the next step without further purification. MS: 609.4 [M+H].

### Step c) potassium ((2R,3S,4R,5R)-4-amino-5-(2,4-dioxo-3,4-dihydropyrimidin-1(2H)-yl)-3-hydroxy-4-methyltetrahydrofuran-2-yl)methyl triphosphate (3c)

The intermediate 2b (0.094 mmol) was dissolved in dry dimethyl formamide (1.5 mL), tributylamine pyrophosphate (0.19 mmol)) was added under nitrogen and the solution stirred overnight at room temperature. The solvent was removed in vacuum and the residue taken into 15% ammonia (12 mL) and stirred at room temperature for 2 h 30 min. Solvents were removed by evaporation and the residue re-dissolved in water containing 5% acetonitrile (4 mL) and washed with DCM (3×2 mL). The organic extracts were discarded, the water layer filtered to remove any insoluble material and the solution concentrated in vacuum. The resulting residue was then purified by preparative HPLC on HyperCarb using a gradient (12 mL/min) from 0% B to 50% B over 12 min (Solvent A: 10 mM ammonium acetate, 95% water, 5% acetonitrile; Solvent B: 10 mM ammonium acetate, 10% water, 90% acetonitrile) to yield an inseparable mixture of the nucleoside phosphates. This mixture was freeze-dried and the residue purified by preparative HPLC on Dionex DNAPac using a gradient (4 mL/min) from 0% B to 60% B over 30 min (Solvent A: 0.05M ammonium bicarbonate, 90% water, 10% acetonitrile; Solvent B: 0.8M ammonium bicarbonate, 90% water, 10% acetonitrile) to yield, after freeze drying twice the desired tri-phosphate in its ammonium salt form (4 mg, 7.5% yield) in 99.2% purity. The salt was dissolved in water containing 5% acetonitrile and passed through Dowex®-K⁺ to afford the tri-phosphate in its potassium salt form. ³¹P-NMR (D₂O) δ: −22.6 (1P, t), −11.5 (1P, d) and −7.20 (1P, d). MS: 498.0 [M+H].

### Example 4

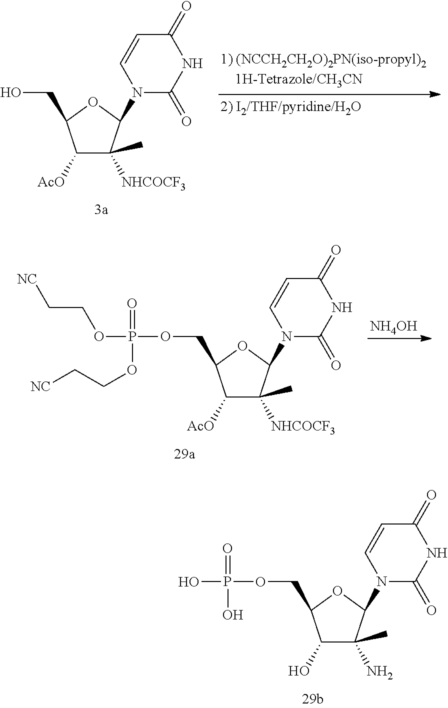

### Step a) (2R,3S,4R,5R)-2-(((bis(2-cyanoethoxy)phosphoryl)oxy)methyl)-5-(2,4-dioxo-3,4-dihydropyrimidin-1(2H)-yl)-4-methyl-4-(2,2,2-trifluoroacetamido)tetrahydrofuran-3-yl acetate (29a)

To a solution of the di-protected nucleoside 3a (0.068 mmol) in acetonitrile (1 mL) under argon were added the bis(2-cyanoethyl)diisopropylphosphoramidite (0.136 mmol) and 1H-tetrazole (0.408 mmol) and the reaction mixture was stirred for 1 h at room temperature. A solution of I₂ (1M in THF/pyridine/water, 7:2:1, 10 eq) was added and to the reaction mixture was stirred for 20 min. The reaction mixture was poured into aqueous saturated Na₂S₂O₃/aqueous saturated NaHCO₃ (1:1, 10 mL) and extracted with DCM. The organic phase was dried, filtered and concentrated and the afforded residue was purified by chromatography on silica eluted with DCM:MeOH (99:1 to 90:10) which gave the title compound (34 mg). MS: 580.2 [M−H]⁻.

### Step b) ((2R,3S,4R,5R)-4-amino-5-(2,4-dioxo-3,4-dihydropyrimidin-1(2H)-yl)-3-hydroxy-4-methyltetrahydrofuran-2-yl)methyl dihydrogen phosphate (29b)

To a solution of the protected nucleotide 29a (0.057 mmol) in methanol (1 mL) was added concentrated NH₄OH (10 mL) and the mixture stirred at room temperature overnight. The reaction mixture was concentrated to dryness and the residue dissolved in 10 mM NH₄Ac buffer/CH₃CN (90:10), filtered and purified by preparative HPLC on HyperCarb using a gradient of 10 mM NH₄OAc, water and CH₃CN, which gave the title compound (13.2 mg). MS: 338.2[M+H]⁺.

### Phosphorylation Method A

### Example 5

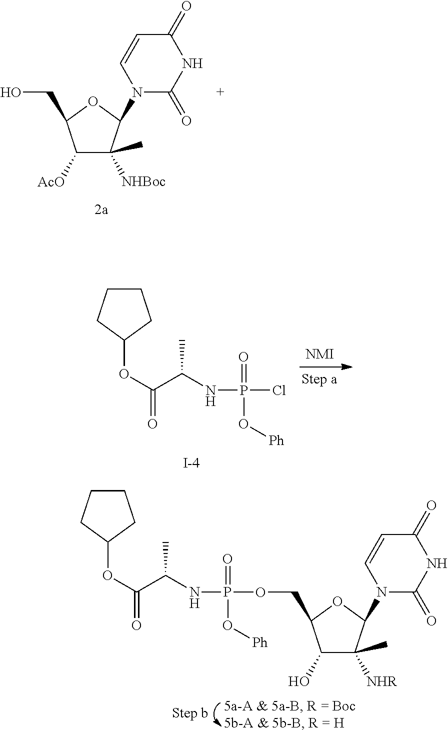

### Step a) (2S)-cyclopentyl 2-(((((2R,3S,4R,5R)-4-((tert-butoxycarbonyl)amino)-5-(2,4-dioxo-3,4-dihydropyrimidin-1(2H)-yl)-3-hydroxy-4-methyltetrahydrofuran-2-yl)methoxy)(phenoxy)-phosphoryl)amino)propanoate (5a-A & 5a-B)

Compound I-4 (232.07 mg, 0.7 mmol) in DCM (1 ml) was added slowly under N₂-atmosphere to a cooled (ice/H₂O/NaCl) solution of nucleoside 2a (100 mg, 0.28 mmol) and N-methylimidazole (96.5 mg, 94 μl, 1.18 mmol) in DCM (5 ml). After 2 h, MeOH (1 ml) was added and the solution was concentrated which gave the title compound as a crude mixture of the two phosphorus diastereomers. The diastereomers were separated on prep. MS: eluted with a gradient of CH₃CN/H₂O (10 mM NH₄OAc): 45/50→50/50, which gave the two diastereomers of the title compound. 1^{st} eluting component 6a-A (42 mg, 23%) and 2^{nd} eluting component 6a-B (51 mg, 28%).

### Step b) (2S)-cyclopentyl 2-(((((2R,3S,4R,5R)-4-amino-5-(2,4-dioxo-3,4-dihydropyrimidin-1(2H)-yl)-3-hydroxy-4-methyltetrahydrofuran-2-yl)methoxy)(phenoxy)phosphoryl)-amino)propanoate (5b-A & 5b-B)

A solution of compound 5a-B (51 mg, 0.08 mmol), in H₂O (2 ml) and acetic acid (3 ml) was stirred at 90° C. for 4 h and then concentrated. The residue was purified by prep. MS: eluted with a gradient of CH₃CN/H₂O (10 mM NH₄OAc) 20/80→40/60, which gave the title compound (32 mg, 74%). MS: 553.3 [M+H]⁺.

Compound 5a-A was deprotected according to the method described for deprotection of compound 5a-B. MS: 553.3 [M+H]⁺.

### Phosphorylation Method B

### Example 6

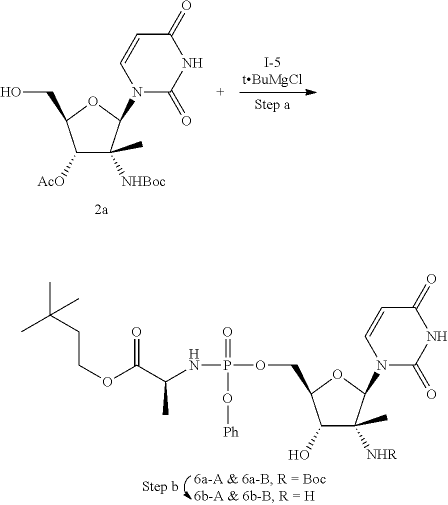

### Step a) (2S)-3,3-dimethylbutyl 2-(((((2R,3S,4R,5R)-4-((tert-butoxycarbonyl)amino)-5-(2,4-dioxo-3,4-dihydropyrimidin-1(2H)-yl)-3-hydroxy-4-methyltetrahydrofuran-2-yl)methoxy)-(phenoxy)phosphoryl)amino)propanoate (6a-A & 6a-B)

Tert. butylmagnesium chloride 1.0 M in THF (0.8 ml) was added under argon during 10 min to a solution of nucleoside 2a (143 mg, 0.4 mmol) in THF (2 ml). The mixture was stirred for 30 minutes and then a solution of (2S)-3,3-dimethylbutyl 2-(((4-nitrophenyl)(phenoxy)-phosphoryl)amino)propanoate (360 mg, 0.8 mmol) in THF (2 ml) was added and the mixture was stirred at room temperature for 48 h. A solution of saturated ammonium chloride was added and the mixture was extracted three times with ethyl acetate. The organic phase was dried with sodium sulphate and concentrated under reduced pressure. The afforded residue was purified by silica gel chromatography eluted with DCM and MeOH which gave a diastereomeric mixture of the title compound (185 mg, 69%). The mixture was separated by HPLC which gave the two diastereomers of the title compound; 1^{st} eluting component 7a-A (57 mg, 21%) and 2^{nd} eluting component, 7a-B (107 mg, 40%). MS: 527.3 [M+1]⁺.

### Step b) (2S)-3,3-Dimethylbutyl 2-(((((2R,3S,4R,5R)-4-amino-5-(2,4-dioxo-3,4-dihydropyrimidin-1(2H)-yl)-3-hydroxy-4-methyltetrahydrofuran-2-yl)methoxy)(phenoxy)-phosphoryl)amino)propanoate (6b-A & 6b-B)

The Boc group was removed from each of the diastereomers 6a-A and 6a-B according to the method described in Example 5 step b, which gave the two diastereomers of the title compound, 6b-A, (30 mg, 64%) and 6b-B, (36 mg, 73%). MS: 569.4 [M+1]⁺.

The following compounds were prepared by phosphorylation of compound 2a with the appropriate phosphorylation agent using Phosphorylation Method A or B, followed by deprotection as described in Example 5 step b.

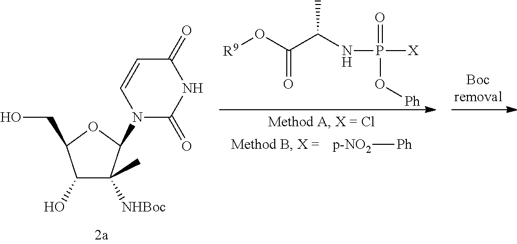

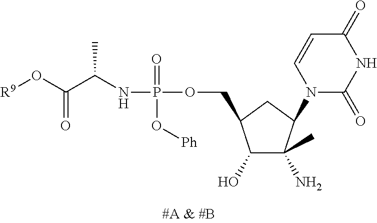

| | | | | | MS |
|---|---|---|---|---|---|
| Name & Ex.# | R⁹ | Method | Yield | | [M + 1]⁺ |
| (2S)-Cycloheptyl 2-(((((2R,3S,4R,5R)-4-amino-5-(2,4- | Cyclo- | B | 7A | 46% | 581.3 |
| dioxo-3,4-dihydropyrimidin-1(2H)-y1)-3-hydroxy-4- | heptyl | | 7B | 48% | 581.3 |
| methyltetrahydrofuran-2- | | | | | |
| yl)methoxy)(phenoxy)phosphoryl)amino)propanoate (7A | | | | | |
| & 7B) | | | | | |
| (2S)-Cyclobutyl 2-(((((2R,3S,4R,5R)-4-amino-5-(2,4- | Cyclobutyl | A | 8A | 81% | 539.2 |
| dioxo-3,4-dihydropyrimidin-1(2H)-yl)-3-hydroxy-4- | | | 8B | 87% | 539.2 |
| methyltetrahydrofuran-2- | | | | | |
| yl)methoxy)(phenoxy)phosphoryl)-amino)-propanoate | | | | | |
| (8A & 8B) | | | | | |
| (2S)-Isopropyl 2-(((((2R,3S,4R,5R)-4-amino-5-(2,4- | Isopropyl | A | 9A | 34% | 527.0 |
| dioxo-3,4-dihydropyrimidin-1(2H)-yl)-3-hydroxy-4- | | | 9B | 53% | 527.0 |
| methyltetrahydrofuran-2- | | | | | |
| yl)methoxy)(phenoxy)phosphoryl)-amino)propanoate | | | | | |
| (9A & 9B) | | | | | |
| (2S)-Cyclooctyl 2-(((((2R,3S,4R,5R)-4-amino-5-(2,4- | Cyclooctyl | B | 36A | 18% | 595.3 |
| dioxo-3,4-dihydropyrimidin-1(2H)-yl)-3-hydroxy-4- | | | 36B | 62% | 595.3 |
| methyltetrahydrofuran-2- | | | | | |
| yl)methoxy)(phenoxy)phosphoryl)amino)propanoate | | | | | |
| (10A & 10B) | | | | | |
| (2S)-Ethyl 2-(((((2R,3S,4R,5R)-4-amino-5-(2,4-dioxo- | Ethyl | A | 11A | 46% | 513.2 |
| 3,4-dihydropyrimidin-1(2H)-yl)-3-hydroxy-4- | | | 11B | 21% | 513.2 |
| methyltetrahydrofuran-2- | | | | | |
| yl)methoxy)(phenoxy)phosphoryl)-amino)propanoate | | | | | |
| (11A & 11B) | | | | | |
| (2S)-Cyclohexyl 2-(((((2R,3S,4R,5R)-4-amino-5-(2,4- | Cyclo- | A | 12A | 22% | 567.4 |
| dioxo-3,4-dihydropyrimidin-1(2H)-yl)-3-hydroxy-4- | hexyl | | 12B | 37% | 567.4 |
| methyltetrahydrofuran-2- | | | | | |
| yl)methoxy)(phenoxy)phosphoryl)-amino)propanoate | | | | | |
| (12A & 12B) | | | | | |
| (2S)-2,2-Dimethylpropyl 2-(((((2R,3S,4R,5R)-4-amino-5- | 2,2- | A | 13A | 59% | 555.1 |
| (2,4-dioxo-3,4-dihydropyrimidin-1(2H)-yl)-3-hydroxy-4- | Dimethyl- | | 13B | 99% | 555.1 |
| methyltetrahydrofuran-2- | propyl | | | | |
| yl)methoxy)(phenoxy)phosphoryl)amino)-propanoate | | | | | |
| (13A & 13B) | | | | | |
| (2S)-2-Propylpentyl 2-(((((2R,3S,4R,5R)-4-amino-5- | 2-Propyl- | A | 14A | 61% | 597.2 |
| (2,4-dioxo-3,4-dihydropyrimidin-1(2H)-yl)-3-hydroxy-4- | pentyl | | 14B | 80% | 597.2 |
| methyltetrahydrofuran-2- | | | | | |
| yl)methoxy)(phenoxy)phosphoryl)amino)propanoate | | | | | |
| (14A & 14B) | | | | | |
| (2S)-Benzyl 2-(((((2R,3S,4R,5R)-4-amino-5-(2,4-dioxo- | Benzyl | A | 15A | 43% | 550.0 |
| 3,4-dihydropyrimidin-1(2H)-yl)-3-hydroxy-4- | | | 15B | 64% | 550.0 |
| methyltetrahydrofuran-2- | | | | | |
| yl)methoxy)(phenoxy)phosphoryl)amino)propanoate | | | | | |
| (15A & 15B) | | | | | |
| (2S)-Methyl 2-(((((2R,3S,4R,5R)-4-amino-5-(2,4-dioxo- | Methyl | A | 16A | 99% | 499.2 |
| 3,4-dihydropyrimidin-1(2H)-yl)-3-hydroxy-4- | | | 16B | 68% | 499.2 |
| methyltetrahydrofuran-2- | | | | | |
| yl)methoxy)(phenoxy)phosphoryl)-amino)propanoate | | | | | |
| (16A & 16B) | | | | | |
| (2S)-Isobutyl 2-(((((2R,3S,4R,5R)-4-amino-5-(2,4-dioxo- | Isobutyl | A | 17A | 62% | 541.1 |
| 3,4-dihydropyrimidin-1(2H)-yl)-3-hydroxy-4- | | | 17B | 56% | 541.1 |
| methyltetrahydrofuran-2- | | | | | |
| yl)methoxy)(phenoxy)phosphoryl)-amino)propanoate | | | | | |
| (17A & 17B) | | | | | |
| (2S)-Pentyl 2-(((((2R,3S,4R,5R)-4-amino-5-(2,4-dioxo- | n-Pentyl | A | 18A | 47% | 555.0 |
| 3,4-dihydropyrimidin-1(2H)-yl)-3-hydroxy-4- | | | 18B | 74% | 555.0 |
| methyltetrahydrofuran-2- | | | | | |
| yl)methoxy)(phenoxy)phosphoryl)-amino)propanoate | | | | | |
| (18A & 18bB) | | | | | |
| (2S)-Butyl 2-(((((2R,3S,4R,5R)-4-amino-5-(2,4-dioxo- | n-Butyl | A | 19A | 86% | |
| 3,4-dihydropyrimidin-1(2H)-yl)-3-hydroxy-4- | | | 19B | 42% | |
| methyltetrahydrofuran-2- | | | | | |
| yl)methoxy)(phenoxy)phosphoryl)-amino)propanoate | | | | | |
| (19A & 19B) | | | | | |
| (2S)-2-Ethylbutyl 2-(((((2R,3S,4R,5R)-4-amino-5-(2,4- | 2-Ethyl- | B | 20A: | 17% | 569.3 |
| dioxo-3,4-dihydropyrimidin-1(2H)-yl)-3-hydroxy-4- | butyl | | 20B: | 30% | 569.3 |
| methyltetrahydrofuran-2- | | | | | |
| yl)methoxy)(phenoxy)phosphoryl)amino)propanoate | | | | | |
| (20A & 20B) | | | | | |
| (2S)-(R)-Sec-butyl 2-(((((2R,3S,4R,5R)-4-amino-5-(2,4- | (R)-Sec- | B | 21A | 75% | 541.3 |
| dioxo-3,4-dihydropyrimidin-1(2H)-yl)-3-hydroxy-4- | butyl | | 21B | 74% | 541.3 |
| methyltetrahydrofuran-2- | | | | | |
| yl)methoxy)(phenoxy)phosphoryl)amino)propanoate | | | | | |
| (21A & 21B) | | | | | |
| (2S)-(R)-Sec-butyl 2-(((((2R,3S,4R,5R)-4-amino-5-(2,4- | (S)-Sec- | B | 22A | 78% | 541.3 |
| dioxo-3,4-dihydropyrimidin-1(2H)-yl)-3-hydroxy-4- | butyl | | 22B | 81% | 541.3 |
| methyltetrahydrofuran-2- | | | | | |
| yl)methoxy)(phenoxy)phosphoryl)amino)propanoate | | | | | |
| (22A & 22B) | | | | | |
| (2S)-(S)-Pentan-2-yl 2-(((((2R,3S,4R,5R)-4-amino-5- | (S)- | B | 23A | 26% | 555.3 |
| (2,4-dioxo-3,4-dihydropyrimidin-1(2H)-yl)-3-hydroxy-4- | Pentan-2- | | 23B | 51% | 555.3 |
| methyltetrahydrofuran-2- | yl | | | | |
| yl)methoxy)(phenoxy)phosphoryl)amino)propanoate | | | | | |
| (23A & 23B) | | | | | |
| (2S)-Propyl 2-(((((2R,3S,4R,5R)-4-amino-5-(2,4-dioxo- | Propyl | B | 24A | 34% | 527.3 |
| 3,4-dihydropyrimidin-1(2H)-yl)-3-hydroxy-4- | | | 24B | 36% | 527.3 |
| methyltetrahydrofuran-2- | | | | | |
| yl)methoxy)(phenoxy)phosphoryl)amino)propanoate | | | | | |
| (24A & 24B) | | | | | |
| (2S)-(R)-Pentan-2-yl 2-(((((2R,3S,4R,5R)-4-amino-5- | (R)- | B | 25A | 18% | 555.3 |
| (2,4-dioxo-3,4-dihydropyrimidin-1(2H)-yl)-3-hydroxy-4- | Pentan-2- | | 25B | 55% | 555.3 |
| methyltetrahydrofuran-2- | yl | | | | |
| yl)methoxy)(phenoxy)phosphoryl)amino)propanoate | | | | | |
| (25A & 25B) | | | | | |
| (2S)-cyclopropylmethyl 2-(((((2R,3S,4R,5R)-4-amino-5- | Cyclo- | B | 33A | 48% | 539.3 |
| (2,4-dioxo-3,4-dihydropyrimidin-1(2H)-yl)-3-hydroxy-4- | propyl- | | 33B | 51% | |
| methyltetrahydrofuran-2- | methyl | | | | |
| yl)methoxy)(phenoxy)phosphoryl)amino)propanoate | | | | | |
| (26A & 26B) | | | | | |
| (2S)-Cyclopentylmethyl 2-(((((2R,3S,4R,5R)-4-amino-5- | Cyclo- | B | 34A | 100% | 567.3 |
| (2,4-dioxo-3,4-dihydropyrimidin-1(2H)-yl)-3-hydroxy-4- | pentyl- | | 34B | 64% | |
| methyltetrahydrofuran-2- | methyl | | | | |
| yl)methoxy)(phenoxy)phosphoryl)amino)propanoate | | | | | |
| (27A & 27B) | | | | | |
| (2S)-Pentan-3-yl 2-(((((2R,3S,4R,5R)-4-amino-5-(2,4- | pentan-3- | B | 35A 28 | mg | |
| dioxo-3,4-dihydropyrimidin-1(2H)-yl)-3-hydroxy-4- | yl | | 35B 58 | mg | |
| methyltetrahydrofuran-2- | | | | | |
| yl)methoxy)(phenoxy)phosphoryl)amino)propanoate | | | | | |
| (28A & 28B) | | | | | |

### Example 29

### Large Scale Synthesis of Compound 20B

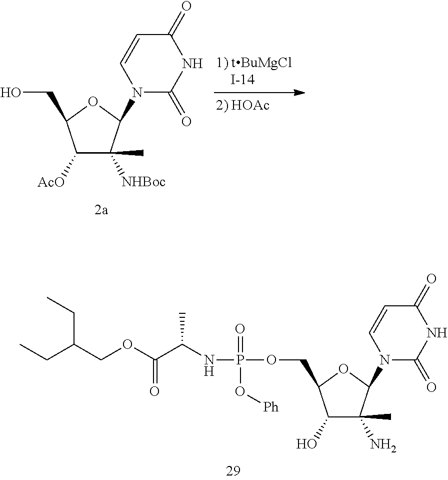 [SMILES: CC(=O)O[C@@H]1[C@@H](CO)O[C@@H](n2ccc(=O)[nH]c2=O)[C@]1(C)NC(=O)OC(C)(C)C ; CCC(CC)COC(=O)[C@H](C)NP(=O)(OC[C@H]1O[C@@H](n2ccc(=O)[nH]c2=O)[C@](C)(N)[C@@H]1O)Oc1ccccc1]

### (2S)-2-ethylbutyl 2-(((((2R,3S,4R,5R)-4-amino-5-(2,4-dioxo-3,4-dihydropyrimidin-1(2H)-yl)-3-hydroxy-4-methyltetrahydrofuran-2-yl)methoxy)(phenoxy)phosphoryl)amino)propanoate (29)

Tert-butylmagnesium chloride (3.02 g, 25.9 mmol) was added under nitrogen at −5° C. to a cold solution of compound 2a (4.4 g, 12.3 mmol) in dry THF (60 mL) and the resulting was slurry stirred for 1 h. A solution of I-14 (6.66 g, 14.8 mmol) in THF (40 mL) was then added and the reaction was allowed to reach room temperature and was stirred for 72 h. The reaction was quenched with ammonium chloride and ice and diluted with EtOAc. The water layer was extracted with EtOAc and the combined organic phases washed with brine, dried over sodium sulfate, filtered and concentrated. The residue was purified by flash chromatography on silica-gel eluting first with hexane/ethyl acetate: 60/40, then with dichloromethane/methanol:97/3 which gave the title compound (3.58 g, 43%).

The afforded residue was dissolved in 60% acetic acid and the solution was stirred at 90° C. for 3.5 h. The solvent was removed under reduced pressure and the crude compound was purified by silica gel column chromatography eluted with DCM and 3 to 12% methanol, which gave the title compound (91%).

### Biological Examples

### Replicon Assay

The compounds of formula I may be examined for activity in the inhibition of HCV RNA replication in a cellular assay aimed at identifying compounds that inhibit a HCV functional cellular replicating cell line, also known as HCV replicons. A suitable cellular assay is based on a bicistronic expression construct, as described by Lohmann et al. (1999), Science vol. 285 pp. 110-113 with modifications described by Krieger et al. (2001), Journal of Virology 75:4614-4624, in a multi-target screening strategy.

The assay utilizes the stably transfected cell line Huh-7 luc/neo (hereafter referred to as Huh-Luc). This cell line harbors an RNA encoding a bicistronic expression construct comprising the wild type NS3-NS5B regions of HCV type 1b translated from an Internal Ribosome Entry Site (IRES) from encephalomyocarditis virus (EMCV), preceded by a reporter portion (FfL-luciferase), and a selectable marker portion (neo^{R}, neomycine phosphotransferase). The construct is bordered by 5′ and 3′ NTRs (non-translated regions) from HCV type 1b. Continued culture of the replicon cells in the presence of G418 (neo^{R}) is dependent on the replication of the HCV RNA. The stably transfected replicon cells that express HCV RNA, which replicates autonomously and to high levels, encoding inter alia luciferase, are used for screening the antiviral compounds.

The replicon cells are plated in 384 well plates in the presence of the test and control compounds which are added in various concentrations. Following an incubation of three days, HCV replication is measured by assaying luciferase activity (using standard luciferase assay substrates and reagents and a Perkin Elmer ViewLux™ ultraHTS microplate imager). Replicon cells in the control cultures have high luciferase expression in the absence of any inhibitor. The inhibitory activity of a compound on luciferase activity is monitored on the Huh-Luc cells, enabling a dose-response curve for each test compound. EC₅₀ values are then calculated, which value represents the amount of the compound required to decrease the level of detected luciferase activity by 50%, or more specifically, the ability of the genetically linked HCV replicon RNA to replicate.

### Enzyme Assay

As may be demonstrated in the replicon assay, the compounds of the invention are metabolised by cellular kinases in target tissues to the 5′-trisphosphate. It is this triphosphate which is believed to be the antivirally active species. The enzyme assay described here example may be used to confirm that compounds of the invention are antivirally active as the 5′-triphosphate metabolite.

The enzyme assay measures the inhibitory effect of triphosphate compounds in an HCV NS5B-21 (21-aminoacid C-terminally truncated version) SPA assay (scintillation proximity assay). The assay is performed by evaluating the amount of radiolabeled ATP incorporated by HCV NS5B-21 into newly synthesized RNA using an heterogeneous biotinylated RNA template.

To determine IC₅₀ values the compounds are tested at various concentrations in a final volume of 100 μl of reaction mixture. The reaction is stopped by addition of 0.5M EDTA solution. The samples are transferred into flashplates precoated with streptavidin. The incorporated radioactivity is quantified using a scintillation counter (Wallac Microbeta Trilux).

### Materials & Supplier

| | |
|---|---|
| Flashplate coated with streptavidin | PerkinElmer Life |
| | Sciences |
| 96 well polypropylene plate | Corning |
| Biotinylated RNA template: with a sequence of | Medprobe |
| 5′-UUU UUU UUU UAG UCA GUC GGC CCG | |
| GUU UUC CGG GCC-3′ and biotinylated at the | |
| 5′-primer end made up to 83 μM in 10 mM Tris- | |
| HCl, 100 mM NaCl, pH = 8.0 | |
| Enzyme: HCV NS5B-21, made up to 500 μg/ml | Replizyme |
| in water. | |
| Nucleotides: GTP, CTP, UTP | Invitrogen |
| Radiolabelled ³H-ATP (cat. no TRK747) | GE Healthcare |
| 0.5M EDTA, pH = 8.0 | Life Technologies |
| Tris-HCl | Sigma |
| MnCl₂ | Sigma |
| Ammonium acetate | Sigma |
| DTT (dithiothreitol) | Sigma |
| CHAPS | Sigma |
| RNase Out (cat. No 10777-019) | Invitrogen |
| DMSO | Carlo Erba |
| | Reactifs - SDS |

### Equipment

| | |
|---|---|
| Wallac Microbeta Trilux | Perkin Elmer Life Sciences |

### Method

### Assay Conditions

| Buffer: 2 0 mM tris-HCl, 100 mM ammonium acetate, 20 | pH 7.5 | |
|---|---|---|
| mM NaCl, 2.5 mM MnCl₂, 10 mM DTT, 2 mM CHAPS, | | |
| RNase Out | | |
| GTP | 50 | μM |
| CTP | 2 | μM |
| UTP | 2 | μM |
| ATP | 2 | μM |
| ³H-ATP (47 Ci/mmol) | 0.5 | μM |
| Template: RNA-H3 | 83 | nM |
| Enzyme: NS5B-21 (500 μg/ml) | 2 | μg/ml |
| Assay volume | 100 | μl |

The assay should include enzyme controls (about four, containing 1 μl DMSO instead of inhibitor) and background control containing all ingredients except template.

Compounds are serially diluted in DMSO on a separate dilution plate to 100× the final desired assay concentrations.

Sufficient reaction mixture for the number of wells to be used is made up according to the table below and 90 μl/well is added to a 96 well polyproylene plate. 1 μl of compound in DMSO from the dilution plate is added to each well, except the enzyme control wells and background control wells to which 1 μl DMSO is added.

### Reaction Mixture

| Component | μl/well |
|---|---|
| 50 mM tris-HCl pH = 7.5 | 40 |
| 1M Ammonium acetate | 10 |
| 1M MnCl₂ | 0.25 |
| 0.5M DTT | 2 |
| 100 mM CHAPS | 2 |
| RNase Out | 0.2 |
| 1 mM GTP | 5 |
| 200 μM CTP + UTP | 2 |
| NS5B-21 500 μg/ml | 0.4 |
| Template: RNA-H3, 83 μM | 0.1 |
| Template buffer: 10 mM tris-HCl, 100 mM NaCl pH = 8.0 | 28.25 |

Prepare an ATP cocktail containing 1.5 μl/well of ³H-ATP (45 Ci/mmol), 2.0 μl/well of 100 μM ATP and 6.5 μl/well of H2O and start the reaction by adding 10 μl/well of this cocktail.

Incubate at 22° C. for 120 min.

Stop the reaction with the addition of 100 μl/well of 0.5M EDTA, pH=8.0.

Transfer 185 μl/well to the streptavidin flash plate.

Incubate the plate over night and read the flash plate in the Microbeta Trilux using the protocol Flash plates H3.

### Treatment of Results

Calculation for Inhibition:

$$\% Inhibition = \frac{CompoundCPM - BackgroundCPM}{AverageEnzymeControlCPM - BackgroundCPM}$$

Background=Reaction buffer without template.

IC₅₀ is determined using Graphpad Prism. Plot Compound concentration in Log versus percentage inhibition. Fit the curve with nonlinear regression to the Log(Inhibitor) versus Response equation.

$$Y = Bottom + \frac{Top - Bottom}{1 + {10}^{( X - log ( {IC}_{50} ) )}}$$

Where Y is % Inhibition, X is log (inhibitor) and top and bottom are the upper and lower limits of the % Inhibition.

### Biological Example 1

The inhibition of HCV replication exhibited by the compounds of the invention were tested in the above described replicon assay. The EC₅₀ values are presented in Table 1.

TABLE 1

| Example | EC₅₀ |
|---|---|
| 1 | >50 |
| 2 | 2 |
| 4 | >50 |
| 5A | 4.7 |
| 5B | 0.47 |
| 6A | 2.3 |
| 6B | 0.49 |
| 7A | 3.0 |
| 7B | 0.19 |
| 8A | 1.9 |
| 8B | 0.8 |
| 9A | 13 |
| 9B | 0.92 |
| 10A | 15 |
| 10B | 0.29 |
| 11A | 6.4 |
| 11B | 0.98 |
| 12A | 0.38 |
| 12B | 0.13 |
| 13A | 2.4 |
| 13B | 0.3 |
| 14A | 1.6 |
| 14B | 0.42 |
| 15A | 3.1 |
| 15B | 0.66 |
| 16A | 5.1 |
| 16B | 2.1 |
| 17A | 2.6 |
| 17B | 0.6 |
| 18A | 1.8 |
| 18B | 0.32 |
| 19A | 1.4 |
| 19B | 0.29 |
| 20A | 1.6 |
| 20B | 0.4 |
| 21A | 5.1 |
| 21B | 0.74 |
| 22A | 21 |
| 22B | 0.66 |
| 23A & B | >50 |
| 24A | 2.9 |
| 24B | 1.5 |
| 25A | 4.7 |
| 25B | 0.38 |
| 26A | 1.7 |
| 26B | 0.76 |
| 27A | 2.2 |
| 27B | 0.38 |
| 28A | 34 |
| 28B | 0.48 |

### Biological Example 2

The nucleotide of Example 3 was tested in the above described enzyme assay and the IC₅₀ value determined to be 13 μM.

### Triphosphate Formation Assay

To estimate the ability of the compounds of the invention to generate the antivirally active triphosphate species, a triphosphate formation assay was conducted. Each compound was tested in triplicates in the assay.

Fresh human plated hepatocytes (Biopredic, France) in 12-well plates were used. Each well was plated with 0.76×10⁶ cells and incubated with a 10 μM DMSO solution of compound (0.1% DMSO) in 1 mL incubation medium in a CO₂ incubator at 37° C. for 6-8 hours. The incubation was stopped by washing each well with 1 mL ice cold Hank's balanced solution, pH 7.2 twice, followed by addition of 0.5 mL ice cold 70% methanol. Immediately after the addition of methanol, the cell-layer was detached from the bottom of the well by a cell scraper and sucked up and down 5-6 times with an automatic pipet. The cell suspension was transferred to a glass vial and stored overnight at −20° C.

The samples, each consisting of various levels of protide, free nucleoside, and mono-, di- and triphosphate were then vortexed and centrifuged at 10° C. for 10 minutes, at 14000 rpm in an Eppendorf centrifuge 5417R. The supernatants were transferred to 2 mL glass vials with insert and subjected to bioanalysis.

### Bioanalysis

An internal standard (Indinavir) was added to each sample and the samples (10 μL injection volume) were analysed on a two column system coupled to a QTRAP 5000 mass spectrometer. The two column system consisted of two binary pumps, X and Y, two switching valves and an autosampler. The two HPLC columns used were a Synergy POLAR-RP 50*4.6 mm, 4 μm particles and a BioBasic AX 50*2.1 mm 5 μm particles. The LC flow rates were 0.4-0.6 mL/min mL/min (the higher flow rate were used in the recondition step).

The HPLC mobile phases for the POLAR-RP column consisted of 10 mmol/L ammonium acetate in 2% acetonitrile (mobile phase A) and 10 mmol/L ammonium acetate in 90% acetonitrile (mobile phase B) and for the BioBasic AX column 10 mmol/L ammonium acetate in 2% acetonitrile (mobile phase C) and 1% ammonium hydroxide in 2% acetonitrile (mobile phase D). The HPLC gradient for pump Y started at 0% mobile phase B and was held for 2 min. During loading phase, the mobile phase went through the POLAR-RP and BioBasic AX column, and prodrug, nucleoside and internal standard were trapped on the POLAR-RP column; whereas the nucleotides (mono-, di- and triphosphates) eluted on to the BioBasic AX column and were trapped there.

In the next step, the flow was switched from the POLAR-RP column to the MS and the mobile phase C switched from pump X to the BioBasic AX column. The compounds on the POLAR-RP column were eluted with a gradient from 0% B up to 100% B in about two minutes and analyzed in positive or negative mode using the multiple reaction monitoring mode (MRM). In the last step the flow from the BioBasic AX column was switched to the MS and the phosphates were eluted with a of about 7 minutes gradient up 50% D) and analyzed in positive or negative mode using MRM. During the last step both columns are reconditioned.

Triphosphate concentration for each compound was then determined by comparison with standard curves. The standard curves were made by analysis of standard samples with known concentrations of triphosphate. The standards were ran in the same matrices as the test samples. Due to variations in phosphorylation levels depending on hepatocyte donor, an internal reference compound is required in each run of the assay in order to enable ranking the results from different runs to each other.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

All documents referred to herein, including patents and patent applications, are incorporated by reference in their entirety.

1. A compound represented by formula I: 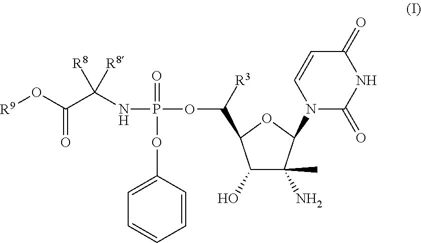
wherein:
R³ is H or CH₃;
R⁸ and R^{8′} are each independently selected from H, C₁-C₆alkyl and benzyl; or R⁸ and R^{8′} together with the carbon atom to which they are attached from a C₃-C₇cycloalkylene group;
R⁹ is C₁-C₁₀alkyl, C₁-C₁₀haloalkyl, C₃-C₇cycloalkyl, benzyl or phenyl, any of which is optionally substituted with 1, 2 or 3 substituents each independently selected from hydroxy, C₁-C₆alkoxy, amino, mono- and di-C₁-C₆alkylamino;
or a pharmaceutically acceptable salt and/or solvate thereof.

2. The compound according to claim 1, wherein R³ is H.

3. The compound according to claim 1, wherein R⁹ is C₁-C₁₀alkyl, C₃-C₇cycloalkyl or benzyl.

4. The compound according to claim 1, wherein R⁹ is C₁-C₆alkyl.

5. The compound according to claim 1, wherein R⁸ is H, and R^{8′} is H or C₁-C₆alkyl.

6. The compound according to claim 1, wherein one of R⁸ and R^{8′} is H and the other is methyl.

7. The compound according to claim 1, wherein R⁸ is H, R^{8′} is C₁-C₃alkyl and R⁹ is C₁-C₆alkyl or C₃-C₇cycloalkyl.

8. The compound according to claim 5, wherein the configuration at the asymmetric carbon atom to which R⁸ and R^{8′} are attached is that of an L-amino acid.

9. A compound according to claim 1, for use as a medicament.

10. The compound according to claim 1, for use in the treatment or prophylaxis of hepatitis C virus infection.

11. A pharmaceutical composition comprising a compound according to claim 1 in association with a pharmaceutically acceptable adjuvant, diluent or carrier.

12. A pharmaceutical composition comprising a compound according to claim 1, further comprising one or more additional other antiviral agent(s).

13. A method for the treatment or prophylaxis of hepatitis C virus infection comprising the administration of a compound according to claim 1.

14. The use of a compound according to claim 1 in the manufacture of a medicament for the treatment or prophylaxis of hepatitis C virus infection.
